(19) United States
(12) Patent Application Publication
(10) Pub. No.: US 2023/0307131 A1
(43) Pub. Date: Sep. 28, 2023

(54) SYSTEMS AND METHODS UTILIZING MACHINE LEARNING TO PREDICT A NEUROLOGICAL CONDITION IN A PERSON

(71) Applicant: Wahrheit, LLC, Lincoln, MA (US)

(72) Inventor: Hans C. Breiter, Lincoln, MA (US)

(21) Appl. No.: 18/119,684

(22) Filed: Mar. 9, 2023

Related U.S. Application Data
(60) Provisional application No. 63/318,321, filed on Mar. 9, 2022.

(51) Int. Cl. G16H 50/20; G16H 10/20; G16H 50/30

(52) U.S. Cl. CPC G16H 50/20; G16H 10/20; G16H 50/30

(57) ABSTRACT

Systems and methods utilize a machine learning classifier to predict diagnoses of conditions or diseases based on a subject’s rating of a plurality of evaluation items, such as pictures, together with demographic information. The pictures may be organized into categories and the subject may provide a positive or negative rating for each picture. The systems and methods may compute relative preference data from the rating data, such as average positive and negative ratings for the evaluation items within each category, information entropy for the categories, and standard deviation for the categories. Plots may be generated of the generated the relative preference data and values for predetermined judgment variables may be derived from the plots. Selected judgment variables and demographic information may be provided to the classifier to produce the prediction for the subject.

### CROSS-REFERENCE TO RELATED APPLICATIONS

The present application claims the benefit of U.S. Provisional Pat. Application Serial No. 63/318,321, filed Mar. 9, 2022, by Hans C. Breiter for SYSTEMS AND METHODS FOR ASSESSING REWARD-AVERSION JUDGMENTS OR PREFERENCES FOR MACHINE LEARNING AND STATISTICAL PREDICTION OF COMMERCIAL, MEDICAL, PSYCHOLOGICAL, MARKETING, RECOMMENDATION, AND FINANCIAL BEHAVIOR, which application is hereby incorporated by reference in its entirety.

### BACKGROUND

### Background Information

Diagnosing cognitive or neurological conditions or disorders, such as SCD, may involve an evaluation by a doctor or clinician as well as medical testing, such as MRIs and blood tests. A subject must find and meet with trained medical professionals. The testing can also be expensive. Furthermore, misdiagnoses still occur.

Subjective Cognitive Decline (SCD), for example, is marked by the ability to perform normally on cognitive assessments despite feeling subjectively impaired. Older individuals appear to be more at risk for Subjective Cognitive Decline (SCD), an early predictor for Alzheimer’s Disease (AD), or dementia preceding mild cognitive impairment (MCI). Even with the usefulness of SCD as a predictor for later cognitive disability, the condition appears to be heterogenous, and consistent quantitative methods for its objective assessment are lacking. Demographic variables appear to impact SCD and research has shown that those with lower income (<$10k), and those failing to complete high school and/or college, were more likely to develop SCD. SCD patients have also been observed to be more impulsive in their decision-making, suggesting a state of rapid forgetting and impaired judgement. In parallel, higher levels of risk aversion (RA) have been predictive of age-related cognitive decline, MCI, dementia, and AD, pointing to the importance of altered judgment in SCD. Multiple studies of structural MRI and multi-model imaging data demonstrate SCD is associated with distinct changes in various brain region volumes. Currently, there is no validated standard for quantitatively assessing SCD, especially one focused on simple judgment and demographic variables.

Research suggests that early intervention is key to slowing the progression of dementia and AD, and SCD has been shown to be predictive of later MCI, dementia, and AD. However, there are no “gold standard” objective methods for assessing SCD, and there appear to be significant demographic vulnerabilities acting as potential confounds (e.g., lesser education). Prior research has revealed relationships between memory disorders (AD, dementia, MCI) and RA. Persons with memory disorders have also been shown to exhibit risky, or impulsive behaviors. Currently, diagnosing SCD and other neurological conditions requires extensive clinical testing with behavioral and clinical assessments that are costly and take significant time. For instance, a subject being assessed for risk of dementia or AD may undergo genetic testing for risk genes, Magnetic Resonance Imaging (MRI) for brain shape and volume changes, or extensive memory and other cognitive assessments that take significant time and are expensive. Even with this extensive testing, the assessments do not have high predictive accuracy for predicting outcomes.

### SUMMARY

Briefly, the present disclosure relates to systems and methods for predicting diagnoses of conditions or diseases based at least in part on a user’s rating of a plurality of evaluation items, such as pictures. The systems and methods may provide a task by which a user views and rates, e.g., positively or negatively, the evaluation items. The evaluation items, e.g., pictures, may be organized into categories. The systems and methods may compute an average (mean) of the positive ratings, e.g., mean approach intensity, for the evaluation items within each category. The systems and methods also may compute an average (mean) of the negative ratings, e.g., mean avoidance intensity, for the evaluation items within each category. The systems and methods may compute an information entropy, such as the Shannon entropy, for the categories. For example, the systems and methods may compute approach Shannon entropy values for each category of evaluation items and avoidance Shannon entropy values for each category. The systems and methods may compute the variance of positive ratings and negative ratings for each category. For example, the systems and methods may compute an approach standard deviation and an avoidance standard deviation for each category of evaluation items.

The systems and methods may generate one or more plots of relationships among the computed data. In some embodiment, the systems and methods may generate one or more of a value function plot, a limit function plot, and a trade-off function plot. The value function plot may plot the relationship of approach and avoidance Shannon entropy against the mean positive and negative ratings. The limit function plot may plot the relationship of approach and avoidance standard deviation against the mean positive and negative ratings. The trade-off function plot may plot the relationship of approach Shannon entropy against avoidance Shannon entropy. The systems and methods may apply one or more curve-fitting tools to derive the function represented by the plots of computed data. For example, the systems and methods may derive a logarithmic function or power function for the value function plot. The systems and methods may derive a quadratic function for the limit function plot. The systems and methods may derive a radial distribution function for the trade-off function plot.

The systems and methods may analyze the one or more plots and/or the functions derived from the one or more plots and compute values for one or more predetermined judgment variables. Exemplary judgement variables that may be computed from the plots or functions include risk aversion, loss resilience, loss aversion, positive offset (ante), negative offset (insurance), positive apex (peak positive risk), negative apex (peak negative risk), positive turning point (reward tipping point), negative turning point (aversion tipping point), positive quadratic area (total reward risk), negative quadratic area (total aversion risk), mean polar angle (reward-aversion tradeoff), polar angle standard deviation (tradeoff range), mean radial distance (reward-aversion consistency), and radial distance standard deviation (consistency range). The systems and methods may construct a user profile for the user that contains the determined values for the one or more predetermined judgement variables. In some embodiments, the systems and methods also may collect demographic information, social information, and/or historical purchase information on the user. Exemplary demographic information includes age, gender, marital status, educational level, income level, home ownership status, number of children, etc. The demographic information may provide a context for the prediction and the systems and methods may include one or more features from the demographic, social, and/or historical purchase information in the user profile.

The systems and methods may provide the user profile to a trained Machine Learning (ML) model/classifier. In some embodiments, the trained ML model/classifier may be a balanced random forest classifier and/or a gaussian mixture model classifier. The user profile may be processed by the trained ML model/classifier, which may generate one or more outputs, e.g., classifications, for the user. The one or more classifications may generate a prediction of a diagnosis for the user. Computing judgment variables based on relative preference theory and utilizing the judgment variables in an ML model/classifier according to the present disclosure provides a quantitative approach for objective detection of SCD, an early marker of dementia and AD in a diverse population, with and without demographic vulnerabilities, e.g., demographic features.

### BRIEF DESCRIPTION OF THE DRAWINGS

The description below refers to the accompanying drawings, of which:

FIG. 1 is a schematic illustration of an example of a machine learning (ML) diagnosis prediction system in accordance with one or more embodiments;

FIGS. 2A-E are partial views of a flow diagram of an example method for predicting a diagnosis in accordance with one or more embodiments;

FIG. 3 is a schematic illustration of an example User Interface (UI) as presented by a rating task in accordance with one or more embodiments;

FIG. 4 is a schematic illustration of an example data structure containing rating data in accordance with one or more embodiments;

FIG. 5 is an illustration of an example value function plot of relative preference data in accordance with one or more embodiments;

FIG. 6 is an illustration of an example limit function plot of relative preference data in accordance with one or more embodiments;

FIG. 7 is an illustration of an example trade-off plot of relative preferences data accordance with one or more embodiments;

FIG. 8 is a schematic illustration of the value function plot of FIG. 5 showing example feature variables in accordance with one or more embodiments;

FIG. 9 is a schematic illustration of the limit function plot of FIG. 6 showing example feature variables in accordance with one or more embodiments;

FIG. 10 is a schematic illustration of the trade-off function plot of FIG. 7 showing example feature variables in accordance with one or more embodiments;

FIG. 11 is a highly schematic illustration of a general, example schema for a Random Forest (RF) or balanced RF Machine Learning (ML) model/classifier in accordance with one or more embodiments;

FIG. 12 is a highly schematic illustration of a general, example schema for a GMM-based ML model/classifier in accordance with one or more embodiments;

FIG. 13 an illustration of a pruned example of the recurrent partitioning and a tabulation of variable importance using Gini scores in accordance with one or more embodiments;

FIG. 14 is a schematic illustration of an example of a pruned decision tree with of a random forest classifier in accordance with one or more embodiments;

FIG. 15 is an illustration of profiles of the tested subjects for a plurality of nodes of the three of FIG. 14 in accordance with one or more embodiments;

FIG. 16 is a schematic illustration of a plurality of iterations of a ML model/classifier in accordance with one or more embodiments;

FIG. 17 is a schematic illustration of an example Random Forest analysis with two major segments for negative credit events in accordance with one or more embodiments;

FIG. 18 is a schematic illustration of an example of a segment from the Random Forest analysis of FIG. 17 in accordance with one or more embodiments;

FIG. 19 is a schematic illustration of another example segment from the Random Forest analysis of FIG. 17 in accordance with one or more embodiments;

FIG. 20 is an illustration of example results from a Multi-Variable Regression (MVLR) analysis of FICO questions in accordance with one or more embodiments;

FIG. 21 is a schematic illustration of a scoring approach in accordance with one or more embodiments;

FIG. 22 is a schematic illustration of an example computer or data processing system in accordance with one or more embodiments; and

FIG. 23 is a schematic diagram of an example distributed computing environment in accordance with one or more embodiments.

### DETAILED DESCRIPTION OF ILLUSTRATIVE EMBODIMENTS

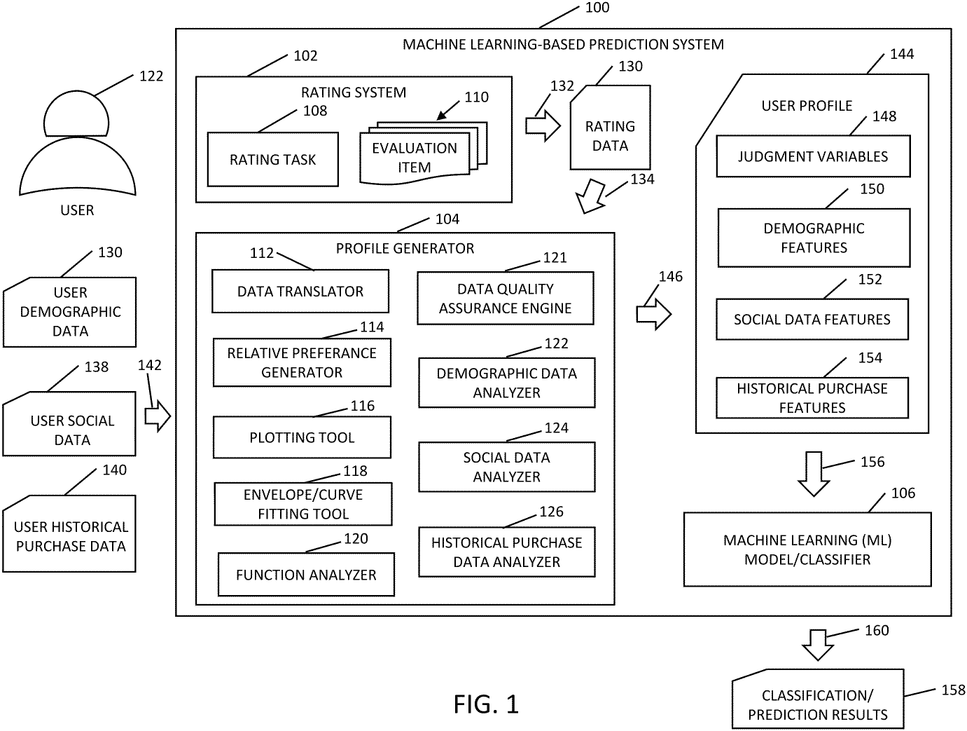

FIG. 1 is a schematic illustration of an example of a machine learning (ML)-based prediction system 100 in accordance with one or more embodiments. The prediction system 100 may include a rating system 102, a profile generator 104, and a ML model and/or classifier 106. In some embodiments, the ML model/classifier 106 is a trained ML model/classifier. The rating system 102 may include a rating task 108 and a plurality of evaluation items indicated at 110. The profile generator 104 may include a data translator 112, a relative preference generator 114, a plotting tool 116, an envelope/curve fitting tool 118, a function analyzer 120, a data quality assurance engine 121, a demographic data analyzer 122, a social data analyzer 124, and a historical purchase analyzer 126. A user 128, which may be a human user, may interact with the rating system 102 producing rating data 130 as indicated by arrow 132. The rating data 130 may be provided to the profile generator 104 as indicated by arrow 134. In some embodiments, the user 128 also may provide user demographic data 136, user social data 138, and/or user historical purchase data 140 to the prediction system 100 as indicated by arrow 142. The profile generator 104 may generate a user profile 144 as indicated by arrow 146. For example, the profile generator 104 may process the rating data 124 and produce one or more judgment variables indicated at 148 that may be included in the user profile 144. The profile generator 104 may also include demographic features 150, social data features 152, and historical purchase features 154 in the user profile 144. The demographic features 150 may be based on the user demographic data 136, the social data features 152 may be based on the user social data 138, and the historical purchase features 154 may be based on the user historical purchase data 140. The user profile 144 may be provided to the ML model/classifier 106 as indicated by arrow 156. The ML model/classifier 106 may generate classification/prediction results 158 as indicated by arrow 160.

In some embodiments, the evaluation items 110 may be pictures that have been well-validated as consistently eliciting an emotional response in the viewer such as pictures that evoke positive or negative feelings in the viewer. In other embodiments, pictures shown to evoke calming or exciting feelings may be selected. In other embodiments, pictures shown to evoke feelings of being in control or under control may be selected. In yet other embodiments, pictures shown to evoke one or more of positive/negative feelings, calming/exciting feelings, or in control/under control feelings may be selected. Suitable pictures for use as the evaluation items 110 may be taken from the International Affective Picture Systems (IAPS), the Ekman facial expression set, or pictures of models vs. non-models, among others. The evaluation items, e.g., pictures, may be organized into categories. Exemplary categories include sports, disasters, cute animals, aggressive animals, nature, e.g., beach vs. mountains, and food. Each category may include at least three pictures for a total of 18 pictures if six categories of pictures are used and preferably each category includes eight pictures for a total of 48 pictures if six categories of pictures are used.

In some embodiments large categories of pictures may be used, such as the eighty pictures used for assessing food for studies of hunger. The pictures need to form a category, or if sound items are used, the sound items need to follow a style of music or framework that distinguishes one group of sounds from another (e.g., coughing vs. laughing vs. crying vs. angry voices). Categorization of pictures or sounds for stimuli can be simple (e.g., shifting the hue of food pictures so the food pictures have fungal coloration vs. normative coloration) or complex (e.g., different acts of violence vs. acts of peace). Each category or group of stimuli acts as a set of exemplars, and the ratings made with them may produce interpretable patterns with entropy variables.

In some embodiments, one or more of the rating system 102, the profile generator 104, and the classifier 106, and/or one or more components thereof, may be implemented through one or more software modules or libraries containing program instructions pertaining to the methods described herein. The software modules may be stored in a memory, such as a main memory, a persistent memory and/or a computer readable media, of one or more data processing machines or devices and executed by one or more processors. Other computer readable media may also be used to store and execute these program instructions, such as non-transitory computer readable media, including optical, magnetic, or magneto-optical media. In other embodiments, one or more of the rating system 102, the profile generator 104, and the classifier 106, and/or one or more components thereof may comprise hardware registers and combinatorial logic configured and arranged to produce sequential logic circuits that implement the methods described herein. In alternative embodiments, various combinations of software and hardware, including firmware, may be utilized to implement the described methods.

A suitable tool for the envelope/curve fitting tool 118 is the Curve Fitting Toolbox from The MathWorks, Inc. of Natick, MA.

Suitable ML models/classifiers 106 include Random Forest (RF) models, including Balanced Random Forest (BRF), and Gaussian Mixture Models (GMMs), among others. RF models may be implemented in the Python programming language using the ‘imblearn’ package and the open access package ‘randomForest’ in the R programming language can be used to train the RF and BRF models on training datasets.

In some embodiments, the rating system 102 may be implemented through software deployed on a device of the user 128, such as a smartphone, tablet, or laptop, among others. For example, the rating system 102 may be implemented as an application (app) that may be downloaded onto the user’s device, e.g., smartphone. The evaluation items 110 may be implemented as digital photos, thereby allowing the user 128 to run the rating task 108 and to rate the evaluation items 110 at his or her convenience.

It should be understood that the evaluation items 110 may take other forms besides pictures. For example, the evaluation items 110 may be videos with or without sound or they may be audio recordings. Furthermore, while digital files may be preferred for the evaluation items 102 to support their presentation on a user device, it should be understood that the evaluation items may be physical items, such as printed pictures or scents.

It should be understood that the ML prediction system 100 as presented in FIG. 1 is intended for illustrative purposes only and that embodiments of the present disclosure may take other forms and be implemented in other ways.

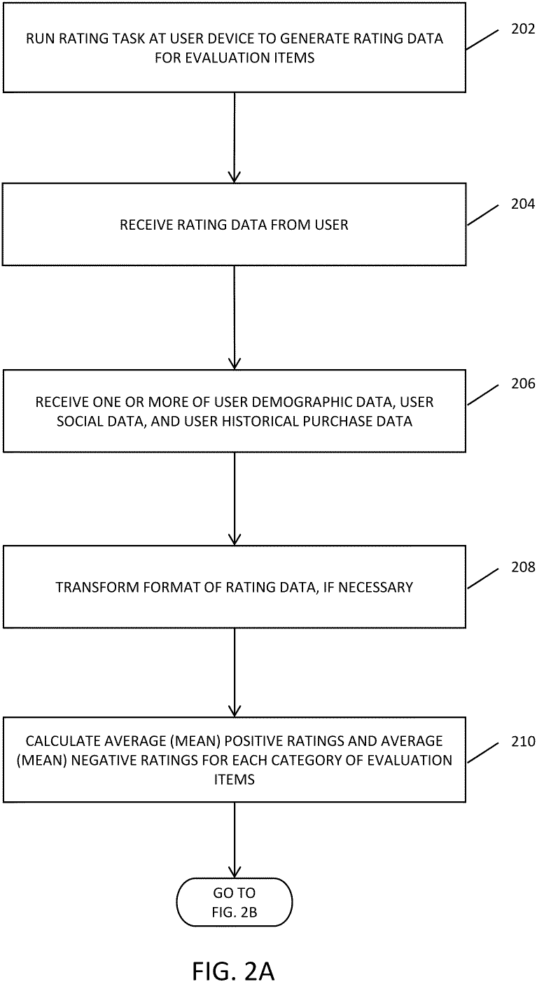

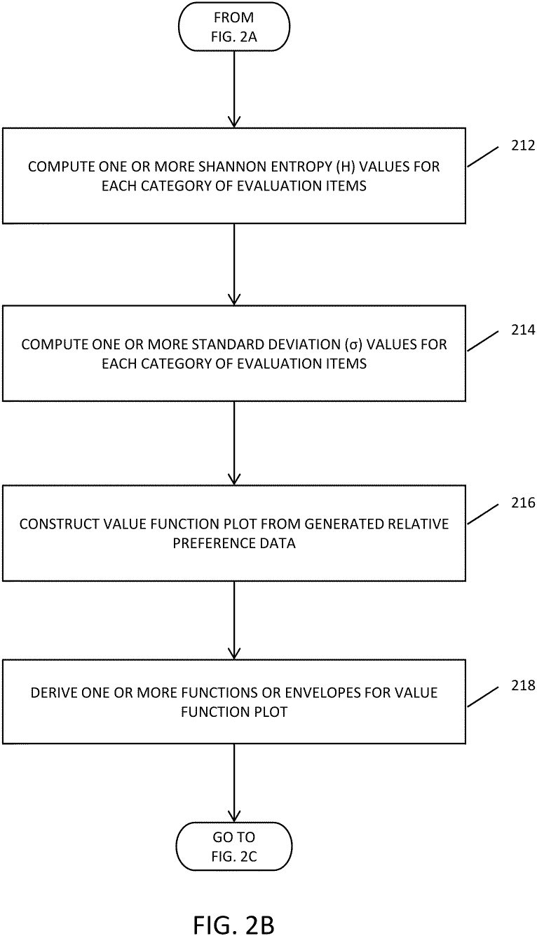

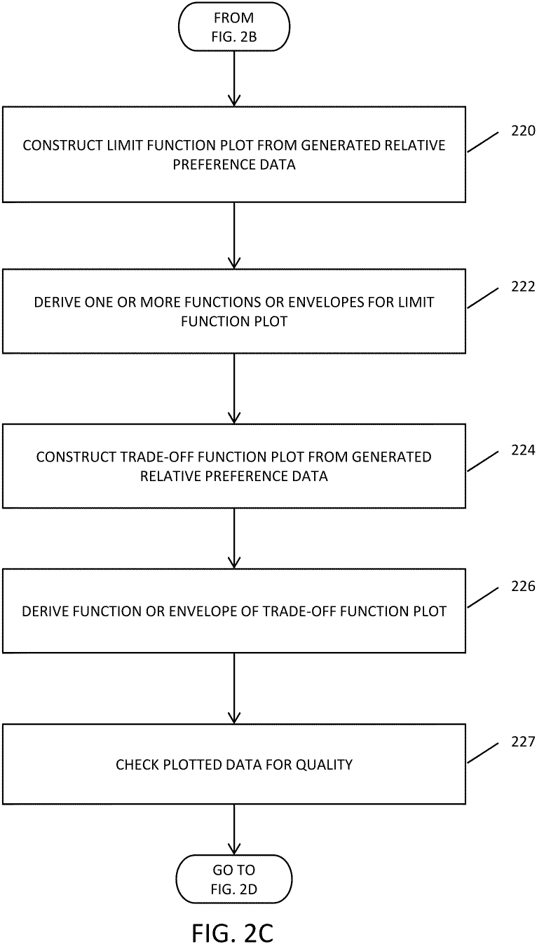

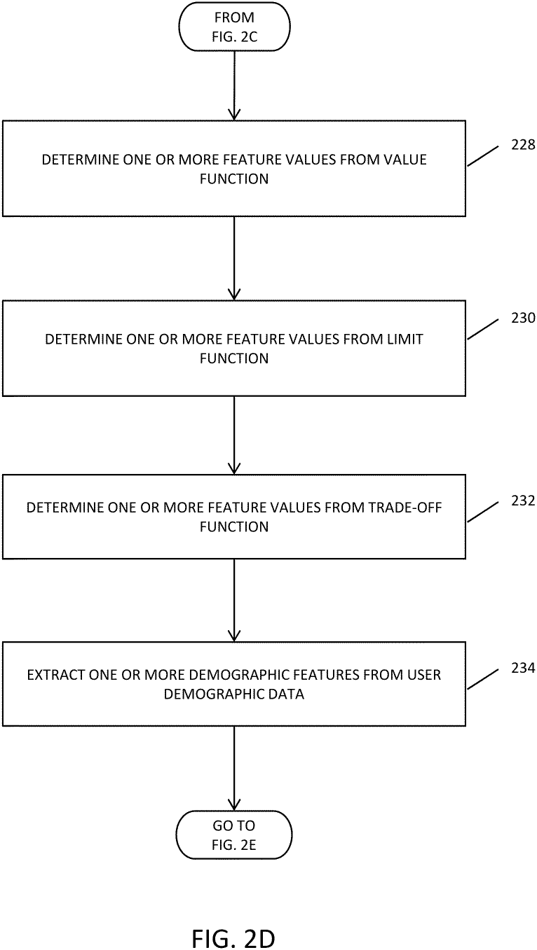

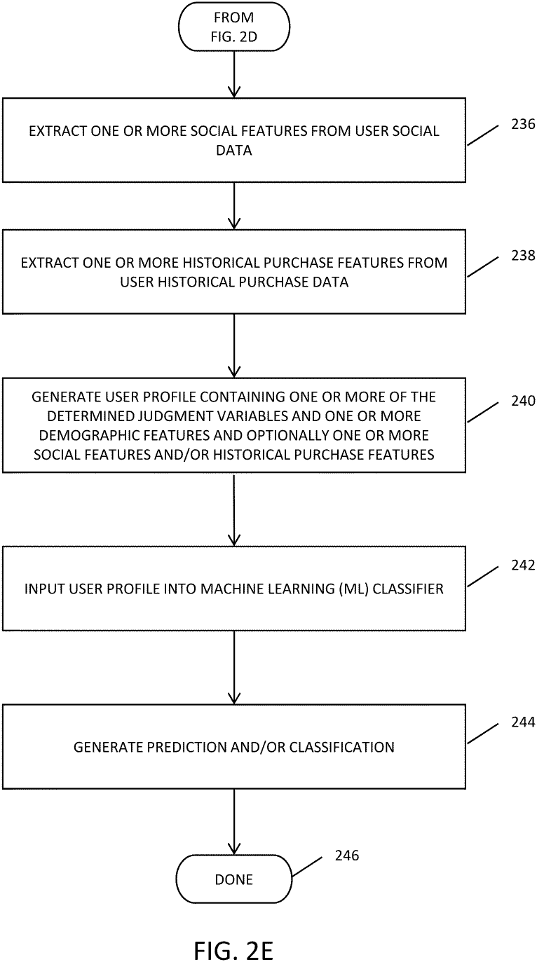

FIGS. 2A-E are partial views of a flow diagram of an example method for predicting a diagnosis of a condition or disease of a human subject in accordance with one or more embodiments. The flow diagrams and description of steps presented herein are for illustrative purposes only. In some embodiments, one or more of the illustrated and/or described steps may be omitted, additional steps may be added, the order of the illustrated steps may be changed, one or more illustrated steps may be subdivided into multiple steps, multiple illustrated steps may be combined into a single step, and/or one or more portions of the flow diagrams and/or descripted steps may be separated into multiple, separate and distinct flow diagrams and/or sequences.

The user 128 may run the rating task 108, e.g., at a user device, as indicated at step 202. The rating task 108 may generate rating data by presenting the evaluation items 110, e.g., a sequence of pictures, to the user 128 and storing picture ratings entered by the user.

In some embodiments, the rating task 108 may be configured to present the following instructions to the user 128 for rating the evaluation items 110:

This task involves looking at pictures and responding how much you like or dislike the image. Please rate each image on an integer scale from -3 (Dislike Very Much) to +3 (Like Very Much). Zero (0) is neutral meaning you have no feelings toward the image either way. Please rate each picture based on your initial emotional response. There are no right or wrong answers. Just respond with your feelings and rate each picture quickly.

In some embodiments, the rating task 108 may not impose a time limit for assigning ratings to each evaluation item 110 or to the rating the entire set of evaluation items 110. As noted, however, the user 128 may be requested to rate each evaluation item 110 as quickly as possible, and the rating task 108 may not permit the user 128 to change their response after selecting a rating. After each rating selection is made, the rating task 108 may present the next evaluation item 110.

The rating task 108 also may present a survey or questionnaire for collecting the user demographic data 136 from the user 128. For example, the questionnaire may obtain the following information from the user: age, gender, race, marital status, educational level, handedness, employment, income level, home ownership status, number of children, age of children, number of grandchildren and great-grandchildren, age of grandchildren and great-grandchildren, number of siblings, health status of siblings, health of the subject, e.g., across common medical problems, ages of parents or if parents are deceased, country of birth, year of immigration if applicable, primary language spoken at home, number of languages spoken, area of education, educational level of children, employment of siblings and parents, county and zip code of birth, county and zip code of current work or residence, political affiliation, attitudes toward smoking, attitudes toward marijuana or drug use, attitudes toward alcohol, pet ownership, activities done during leisure time, among other demographic information. In some embodiments, the one or more surveys or questionnaires may also obtain information about medical condition and/or social condition, such as perceived loneliness using a self-report of a Likert-like scale. In some embodiments, the one or more surveys or questionnaires may obtain information on depression symptoms, for example using the Patient Health Questionnaire (PHQ-9), and behavioral health disorders, e.g., internalizing or externalizing psychiatric disorders, substance use disorders, or crime/violence problems, for example from the GaIN-SS short screen assessment.

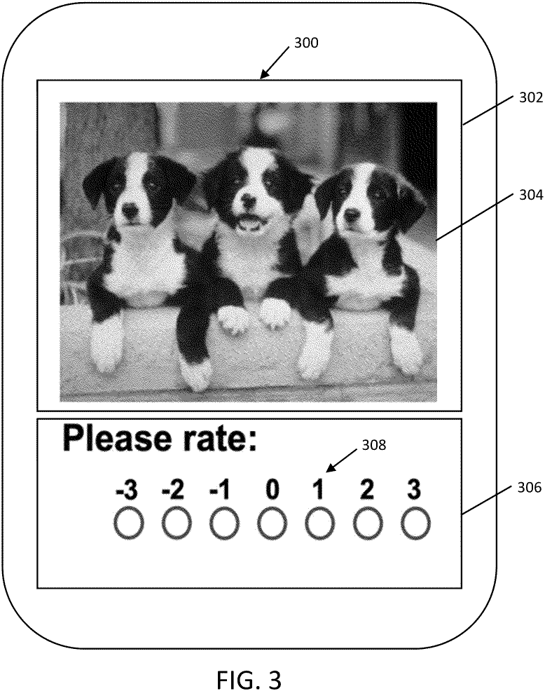

FIG. 3 is a schematic illustration of an example User Interface (UI) 300 as presented by the rating task 108 in accordance with one or more embodiments. The UI 300 may include a picture area 302 in which a picture 304 may be presented. The UI 300 also may include a rating area 306 in which one or more rating widgets or elements, such as rating element 308, may be presented. The user 128 may enter a rating, e.g., an integer between -3 and +3, for the picture 304 through the rating element 308 and the rating may be captured and stored by the rating task 108.

The rating task 108 may generate one or more data structures containing the rating data 130 and the user demographic data 136.

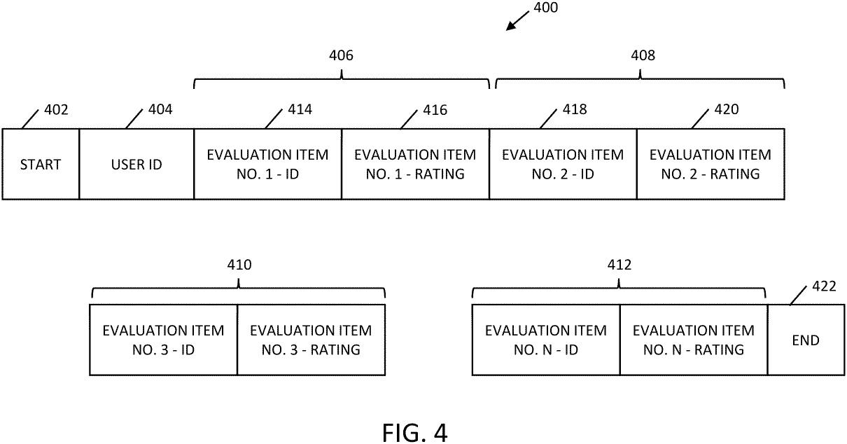

FIG. 4 is a schematic illustration of an example data structure 400 containing the rating data 130 in accordance with one or more embodiments. The data structure 400 is organized into a plurality of elements, such as fields or records, including a start field 402 that may mark the start of the data structure 400. The data structure 400 also may include a participant identifier (ID) field 404 that may store the user’s name, login ID, or other identifier associated with the user 128. The data structure 400 also may include an evaluation item area for each evaluation item 110, such as evaluation item areas 406, 408, 410 and 412, which correspond to evaluation items 1, 2, 3 and N. The evaluation items areas 406, 408, 410 and 412 may include an item ID field that identifies the particular evaluation item 110 and a rating field that contains the rating entered by the user 128 for the evaluation item 110. For example, area 406 may include an item ID field 414 and a rating field 416, area 408 may include an item ID field 418 and a rating field 420, and so on. The data structure 400 also may include an end field 422 that marks the end of the data structure 400.

It should be understood that the data structure 400 is for illustrative purposes and that data structures having additional, fewer or other fields or records may be used with the present disclosure. For example, in some embodiments, one or more error correction codes may be included in the data structure 400. It should also be understood that other forms of data structures or storage elements may be used to store the rating data 130 and/or the user demographic data 136, such as files, objects, containers, frames, messages, etc.

The profile generator 104 may receive the rating data 130 generated by the user 128, as indicated at step 204. The profile generator 104 also may receive the user demographic data 136, as indicated at step 206.

In some embodiments, the data translator 112 may transform the rating data 130 to a form suitable for further processing by the profile generator 104, as indicated at step 208. For example, in some cases, the rating task 108 may be configured to receive star ratings for the evaluation items. For example, the user 128 may be requested to give each evaluation item, e.g., picture, a one to five star rating. In other embodiments, the profile generator 104 may receive star ratings. For example, the profile generator 104 may access previously rated evaluation items, such as purchased items, media items, etc. The data translator 112 may transform the star ratings entered by the user 128 for the evaluation items 110 as follows:

| Star Rating | Numeric Rating |
|---|---|
| 1 | -2 |
| 2 | -1 |
| 3 | 0 |
| 4 | +1 |
| 5 | +2 |

It should be understood that the data translator 112 may apply other data translations schemes to the star ratings.

The relative preference generator 114 may generate relative preference data, such as average (mean) ratings, information entropy, standard deviation, etc., for the categories of evaluation items 110 from the rating data 130.

### Mean Positive and Negative Ratings

The relative preference generator 114 may compute average (mean) positive ratings and average (mean) negative ratings for the categories of evaluation items based on the rating data as indicated at step 210. For example, suppose the user 128 entered various positive ratings for one picture category, e.g., cute animals, and various negative ratings for another picture category, e.g., aggressive animals. The relative preference generator 114 may compute the average positive rating (K+) for the cute animals category and the average negative rating (K₋) for the aggressive animals. If the user 128 entered both positive and negative ratings for the pictures of a given category, e.g., sports, the relative preference generator 114 may compute both an average positive rating (K+) and an average negative rating (K₋) for that category.

### Shannon Entropy

The relative preference generator 114 may generate one or more Shannon entropy values, such as an approach Shannon entropy value (H+) and an avoidance Shannon entropy value (H₋), for each category of evaluation items 110, as indicated at step 212 (FIG. 2B).

The relative preference generator 114 may compute approach Shannon entropy value (H₊) as follows:

$${H}_{+} = {\sum }_{i = 1}^{N} {p}_{+ i} ∗ l o {g}_{2} \left(1 / {p}_{+ i}\right)$$

where,
i is the current evaluation item,
N is the total number of evaluation items in the category for which entropy is being computed, and
p₊ᵢ is the relative proportion of positive responses for the i^{th} evaluation item in the category.

The relative approach probability for the i^{th} evaluation item corresponding to a given evaluation item in the category may be computed as follows:

$${p}_{+ i} = \frac{{a}_{+ i}}{{\sum }_{j = i}^{N} {a}_{+ j}}$$

where,
a₊ᵢ is the positive rating for i^{th} evaluation item, and
N is the total number of evaluation items in the category.

The relative preference generator 114 may compute avoidance Shannon entropy value (H₋) as follows:

$${H}_{-} = {\sum }_{i = 1}^{N} {p}_{- i} ∗ l o {g}_{2} \left(1 / {p}_{- i}\right)$$

where,
i is the current evaluation item,
N is the total number of evaluation items in the category for which entropy is being computed, and
p₋ᵢ is the relative proportion of avoidance responses for the i^{th} evaluation item in the category.

The relative avoidance probability for the i^{th} evaluation item may be computed as follows:

$${p}_{- i} = \frac{{a}_{- i}}{{\sum }_{j = i}^{N} {a}_{- j}}$$

where,
a₋ᵢ is the negative rating for i^{th} evaluation item, and
N is the total number of evaluation items in the category.

It should be understood that other techniques or equations may be employed to compute the approach and avoidance Shannon entropy values or other entropy values. For example, another way of computing approach and avoidance entropy values is given by:

$${H}_{+} = {\sum }_{i = 1}^{N} {p}_{+ i} / log {p}_{+ i}$$

$${H}_{-} = {\sum }_{i = 1}^{N} {p}_{- i} / log {p}_{- i}$$

In some embodiments, the relative preference generator 114 may be configured to compute only an approach Shannon entropy value or only an avoidance Shannon entropy value for each category of evaluation items 110.

In some embodiments, the relative preference generator 114 may be configured to compute other entropy values, such as entropy values based on second or third order models. A suitable equation for computing entropy of a second order model is given by:

$$H = {\sum }_{i = 1}^{m} {p}_{i} {\sum }_{j = 1}^{m} {P}_{j i} log {P}_{j i}$$

where Pᵢⱼ is the conditional probability that the present item is the j^{th} item in the set given that the previous item is the i^{th} item.

A suitable equation for computing entropy of a third order model is given by:

$$H = {\sum }_{i = 1}^{m} {p}_{i} {\sum }_{j = 1}^{m} P j i {\sum }_{j = 1}^{m} {P}_{k j i} log {P}_{k j i}$$

where Pₖⱼᵢ is the conditional probability that the present item is the k^{th} item in the set given that the previous item is the j^{th} item and the one before that is the i^{th} item.

### Standard Deviation

The relative preference generator 114 may generate one or more standard deviation values, such as an approach standard deviation (σ+) value and an avoidance standard deviation (σ-) value for each category of evaluation items 110, as indicated at step 214.

The relative preference generator 114 may compute the approach standard deviation value (σ+) as follows.

$${σ}_{+} = \sqrt{\frac{1}{N} {\sum }_{i = 1}^{N} {\left({R}_{+ i} - {K}_{+}\right)}^{2}}$$

where
σ+ is the approach standard deviation,
N is the total number of evaluation items in the category for which the standard deviation is being computed,
R₊ᵢ is the positive rating for the i^{th} evaluation item of the category, and
K+ is the average (mean) positive rating of the evaluation items in the category.

The relative preference generator 114 may compute the avoidance standard deviation, σ₋, for each category in a similar manner.

As described, the relative preference generator 114 may generate the following relative preference data for the categories of evaluation items 110:

{mean positive rating (K₊), mean negative rating (K₋), approach entropy (H+), avoidance entropy (H₋), approach standard deviation (σ₊), and avoidance standard deviation (σ₋)}

The relative preference data may be generated from the rating data 130, such as numeric ratings or star ratings that quantify (i) a user’s decision-making regarding approach, avoidance, indifference, and uncertain/inconsistent responses to evaluation items, and (ii) judgments that determine the magnitude of approach and avoidance to the items.

In some embodiments, the plotting tool 116 may generate one or more plots of the generated relative preference data, such as a value function plot, a limit function plot, and/or a trade-off plot.

### Value Function Plot

The plotting tool 116 may construct a value function plot from the generated relative preference data as indicated at step 216. A value function plot may be a plot of approach entropy (H+) versus average (mean) positive rating (K+), and avoidance entropy (H₋) versus average (mean) negative rating (K₋).

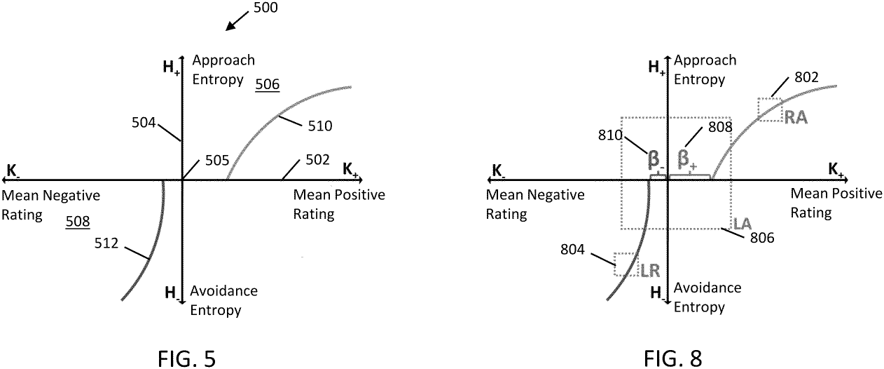

FIG. 5 is an illustration of an example value function plot 500 of relative preference data generated for the user 128 in accordance with one or more embodiments. The value function plot 500 includes an x-axis 502 and a y-axis 504 that intersect at origin 505. The x-axis 502 represents average (mean) rating with the positive side of the x-axis 502 representing average (mean) positive rating and the negative side of the x-axis 502 representing average (mean) negative rating. The y-axis 504 of the value function plot 500 represents the Shannon entropy, with the positive side of the y-axis 504 representing approach Shannon entropy (H+) and the negative side of the y-axis 504 representing avoidance Shannon entropy (H₋).

As described, for each category of evaluation items 110, the relative preference generator 114 may compute a {H+, K+} value pair and/or a {H₋, K₋} value pair. For each category of evaluation items 110, these two value pairs may plotted on the value function plot 500. That is, for each category of evaluation items 110, there may be two points that are plotted, one point in an H+/K+ quadrant 506 of the value function plot 500, and the other point in a H₋/K₋ quadrant 508.

The envelope/curve fitting tool 118 may evaluate the data plotted in the H+/K+ quadrant 506 of the value function plot 500 to determine an approach boundary envelope 510 as indicated at step 218. The approach boundary envelope 510 as determined by the envelope/curve fitting tool 118 may follow a power function given by:

$${\text{H}}_{+} \left({\text{K}}_{+}\right) = {\text{bK}}_{+} {}^{\text{a}}$$

where a and b are coefficients of the power function and are determined by the envelope/curve fitting tool 118.

Alternatively, the envelope/curve fitting tool 118 may determine that the approach boundary envelope 510 may be approximated by a logarithmic function given by:

$${\text{H}}_{+} \left({\text{K}}_{+}\right) = {\text{a*log}}_{10} \left({\text{K}}_{+}\right) + \text{b}$$

where a and b are coefficients of the logarithmic function and are determined by the envelope/curve fitting tool 118.

The envelope/curve fitting tool 118 also may evaluate the data plotted in the H₋/K₋ quadrant 508 of the value function plot 500 to determine an avoidance boundary envelope 512 as also indicated at step 218. The avoidance boundary envelope 512 also may follow a power function given by:

$${\text{H}}_{-} \left({\text{K}}_{-}\right) = {\text{bK}}_{-} {}^{\text{a}}$$

where a and b are coefficients of the power function and are determined by the envelope/curve fitting tool 118.

Alternatively, the envelope/curve fitting tool 118 may determine that the avoidance boundary envelope 512 may be approximated by a logarithmic function given by:

$${\text{H}}_{-} \left({\text{K}}_{-}\right) = {\text{a*log}}_{10} \left({\text{K}}_{-}\right) \text{+ b}$$

where a and b are coefficients of the logarithmic function and are determined by the envelope/curve fitting tool 118.

### Limit Function Plot

The plotting tool 116 may construct a limit function plot from the generated relative preference data as indicated at step 220 (FIG. 2C). The limit function plot may plot the approach standard deviation (σ₊) versus average (mean) positive rating (K+), and avoidance standard deviation (σ₋) versus average (mean) negative rating (K₋).

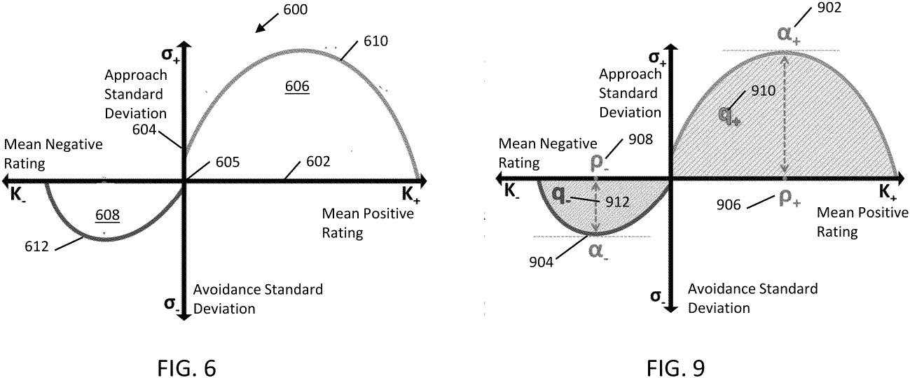

FIG. 6 is an illustration of an example limit function plot 600 of relative preference data generated for the user 128 in accordance with one or more embodiments. The limit function plot 600 has an x-axis 602 and a y-axis 604 that intersect at origin 605. The x-axis 602 represents average (mean) rating with the positive side of the x-axis 602 representing average (mean) positive rating, and the negative side of the x-axis 602 representing average (mean) negative rating. The y-axis 604 represents the standard deviation, with the positive side of the y-axis 604 representing standard deviation for approach, and the negative side of the y-axis 604 representing standard deviation for avoid.

As described, for each category of evaluation items 110, the relative preference generator 114 may compute a {σ₊, K₊} value pair and a {σ₋, K₋} value pair. For each category of evaluation items 110, these two value pairs may be plotted on the limit function plot 600. That is, for each category of evaluation items 110, there may be two points that are plotted, one point in an σ₊/K₊ quadrant 606 of the limit function plot 600, and the other point in a σ₋/K₋ quadrant 608.

The distance a value pair is away from the x-axis 602, i.e., the magnitude of the standard deviation, indicates how difficult the decision was for the user 128 to either approach or avoidance to the respective category of evaluation items 110.

The envelope/curve fitting tool 118 may evaluate the data plotted in the σ₊/K₊ quadrant 606 of the limit function plot 600 to determine an approach boundary envelope 610 as indicated at step 222. The approach boundary envelope 610 as determined by the envelope/curve fitting tool 118 may follow a quadratic function given by:

$${σ}_{+} \left({\text{K}}_{+}\right) = {\text{aK}}_{+} {}^{2} + {\text{bK}}_{+} + \text{c}$$

where a, b, and c are coefficients of the quadratic function as determined by the envelope/curve fitting tool 118.

The envelope/curve fitting tool 118 may evaluate the data plotted in the σ₋/K₋ quadrant 608 of the limit function plot 600 to determine an avoidance boundary envelope 612 as also indicated at step 222. The avoidance boundary envelope 612 as determined by the envelope/curve fitting tool 118 also may follow a quadratic function given by:

$${σ}_{-} \left({\text{K}}_{-}\right) = {\text{aK}}_{-} {}^{2} + {\text{bK}}_{-} + \text{c}$$

where a, b, and c are coefficients of the quadratic function as determined by the envelope/curve fitting tool 118.

The coefficients or fitting parameters for the approach and avoidance envelopes 610, 612 may be different, e.g., avoidance saturation may be more compact than approach saturation, although the general description of the envelopes may be similar.

Alternatively, the boundary envelopes 606, 608 for the limit function plot 600 may be given by:

$${σ}_{+} = a {K}_{+}^{b} cos \left(\frac{{K}_{+}}{c}\right)$$

$${σ}_{-} = a {K}_{-}^{b} cos \left(\frac{{K}_{-}}{c}\right)$$

where, a, b, and c are coefficients.

### Trade-off Function Plot

The plotting tool 116 may construct a trade-off function plot from the generated relative preference data as indicated at step 224. The trade-off plot may plot the approach entropy (H+) versus the avoidance entropy (H₋) computed for the categories of evaluation items 110.

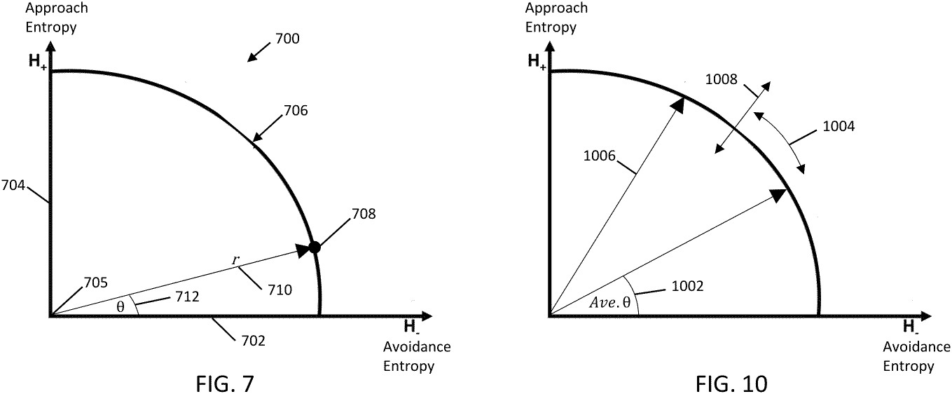

FIG. 7 is an illustration of an example trade-off plot 700 of relative preference data computed for the user 128 in accordance with one or more embodiments. The trade-off plot 700 includes an x-axis 702 and a y-axis 704 that intersect at origin 705. The x-axis 702 represents avoidance Shannon entropy (H₋) values while the y-axis 704 represents approach Shannon entropy (H+) values. As described, for each category of evaluation items 110, the relative preference generator 114 may compute an {H+, H₋} value pair.

The envelope/curve fitting tool 118 may evaluate the data plotted in the trade-off function plot 700 to determine a best-fit curve, such as arc 706, through the {H+, H₋} value pairs as indicated at step 226. The envelope or arc 706 as determined by the envelope/curve fitting tool 118 may be given by:

$$r = \sqrt{\left({H}_{+}^{2} + {H}_{-}^{2}\right)}$$

Thus, each {H+, H₋} value pair has polar coordinates, e.g., {r, θ}, where r is the radial distance from the origin 705 of the trade-off plot 700 to the respective {H+, H₋} value pair, and θ is the angle of the radial, r, from the x-axis 702. Considering a data point, i.e., an entropy pair, 708, for example, there is a radius, r, 710 and a polar angle, θ, 712. Thus, in addition to computing a {H+, H₋} value pair for each evaluation item 110, the function analyzer 120 may compute a polar coordinate pair.

The envelope or arc 708 provides an indication of the relative preference ordering of the categories of evaluation items 110 by the user 128. Specifically, values for evaluation items 110 that appear toward the upper left of the plot 700, which have high approach Shannon entropy (H+) values, are preferred by the user 128 while values for the evaluation items 110 that appear toward the lower right portion of the plot 700, which have high avoidance Shannon entropy (H₋) values, are disliked by the user 128.

It should be understood that one or more preference Trade-off plots may be generated based on other relative preference data besides Shannon entropy. For example, the plotting tool 116 may be configured to generate an SNR Trade-off plot.

In some embodiments, the data quality assurance engine 121 may assess the data from one or more of the value function, limit function, or trade-off function plots, as indicated at step 227. For example, the data quality assurance engine 121 may assess whether R² values > 0.80 where R² is a statistical measure of goodness of fit. In some embodiments, the data quality assurance engine 121 may discard data values whose R² is < 0.80 prior to deriving functions and/or performing curve fitting. In some embodiments, the data quality assurance engine 121 may discard graphical fits that are not concave relative to the x-axis and do not following the concave curvature of group data.

It should be understood that the plots 500, 600, and 700 are shown for explanation purposes. The profile generator 104 may store the data represented by the plots 500, 600, and 700 in one or more data structures, such as files, objects, containers, etc., within one or more memories of a data processing device. In other embodiments, the plotting tool 116 may present one or more of the plots 500, 600, and 700 to a user of the prediction system 100. For example, the plotting tool 116 may present one or more of the plots 500, 600, and 700 on a display of a data processing device.

### Judgment Variables

The function analyzer 120 may derive one or more feature values, e.g., judgment variables, from the value function plot 500 and/or compute the one or more judgment variables from the derived function, as indicated at step 228 (FIG. 2D).

FIG. 8 is a schematic illustration of the value function plot 500 of FIG. 5 showing example feature values extracted from the plot 500 in accordance with one or more embodiments.

In some embodiments, the function analyzer 120 may compute one or more of the following feature values from the value function plot or curve:

1) A risk aversion (RA) value 802, which is a ratio of a second derivative of the value function curve to a first derivative of the value function, which is also a curve, and a predetermined quantity of the approach ratings, such as K+ = 1.5. The RA may represent a measure of the degree to which the user 128 prefers a likely reward in comparison to a better more uncertain reward.

2) A loss resilience (LR) value 804, which is an absolute value of the ratio of the second derivative of the value function to the first derivative of the value function, which is also a curve, and a predetermined quantity of the avoidance ratings, such as K₋ = -1.5. The LR may represent a degree to which the user 128 prefers to lose a small defined amount in comparison to losing a greater amount with more uncertainty associated with this loss.

3) A loss aversion (LA) value 806, which is an absolute value of a ratio of a linear regression slope of a logarithm of the avoidance ratings versus a logarithm of the avoidance entropy values to a linear regression slope of a logarithm of the approach ratings versus a logarithm of the approach entropy values. The LA may measure the degree to which the user 128 outweighs losses to gains.

4) A positive offset or ante (β+) value 808, which is the positive rating (K+) value when setting approach entropy (H+) = 0. The positive offset may measure the ante the user 128 needs to engage in a game of chance.

5) A negative offset or insurance (β-) value 810, which is negative rating (K₋) value when setting avoidance entropy (H₋) = 0. The negative offset may measure how much insurance the user 128 might need against bad outcomes.

It should be understood that the plotting tool 116 may generate one or more value function plots may be generated based on other relative preference data besides Shannon entropy. For example, the plotting tool 116 may be configured to generate one or more SNR value function plots.

The function analyzer 120 may derive one or more feature values, e.g., judgment variables, from the limit function plot 600 and/or compute the one or more feature judgment variables from the derived function, as indicated at step 230.

FIG. 9 is a schematic illustration of the limit function plot 600 of FIG. 6 showing example feature values extracted from the plot 600 in accordance with one or more embodiments.

In some embodiments, the function analyzer 120 may compute the following features from the limit function plot or curve:

1) A peak positive risk or positive apex (α+) value 902, which is the value of the approach standard deviation (σ+) when the derivative of the approach standard deviation to the derivative of the positive ratings

$$\left(\frac{d {σ}_{+}}{d {K}_{+}}\right)$$

is zero. The positive apex may model where increases in positive value transition from a relationship with increased risk to a relationship with decreased risk.

2) A peak negative risk or negative apex (α-) value 904, which is the value of the avoidance standard deviation (σ-) when the derivative of the avoidance standard deviation to the derivative of the negative ratings

$$\left(\frac{d {σ}_{-}}{d {K}_{-}}\right)$$

is zero. The negative apex may represent a maximum variance for avoidance behavior.

3) A positive turning point or reward tipping point (ρ₊) value 906, which is the positive rating (K+) value when the derivative of the approach standard deviation to the derivative of the approach ratings

$$\left(\frac{d {σ}_{+}}{d {K}_{+}}\right)$$

is zero. The positive turning point represents the rating intensity with maximum variance for approach behavior, potentially when the user 128 decides to approach a goal-object.

4) A negative turning point or aversion tipping point (ρ₋) value 908, which is the negative rating (K₋) value when the derivative of the avoidance standard deviation to the derivative of the negative ratings

$$\left(\frac{d {σ}_{-}}{d {K}_{-}}\right)$$

is zero. The negative turning point represents therating intensity with maximum variance for avoidance behavior, potentially when the user 128 decides to avoid a goal-object.

5) A positive quadratic area or total reward risk (q₊) value 910, which is the area under the limit function for the positive ratings (K+) and the approach standard deviation values (σ₊). The positive quadratic area represents the relationship between positive ratings and approach standard deviation and measures the amount of value the user 128 associates to positive stimuli.

6) A negative quadratic area or total aversion risk (q₋) value 912, which is the area under the limit function for the negative ratings (K₋) and the avoidance standard deviation values (σ₋). The negative quadratic area represents the relationship between negative ratings and avoidance standard deviation and measures the aversive value the user 128 associates to negative stimuli.

The function analyzer 120 may derive one or more feature values, e.g., judgment variables, from the trade-off function curve 700 and/or compute the one or more judgment variables from the derived function, as indicated at step 232.

FIG. 10 is a schematic illustration of the trade-off function plot 700 of FIG. 7 showing example feature values extracted from the plot 700 in accordance with one or more embodiments.

In some embodiments, the function analyzer 120 may compute the following feature values from the trade-off function plot or curve:

1) A mean polar angle or reward-aversion tradeoff (θ) value 1002, which is the average (mean) of the polar angles of the data points in the (H₋, H+) plane. The mean polar angle may measure the mean balance for the entropies or patterns in approach vs. avoidance behavior. It may signify the balance in approach and avoidance judgments across the categories of evaluation items 110.

2) A polar angle standard deviation or tradeoff range (σ_{θ}) value 1004, which is the standard deviation of the polar angles of the points in the (H₋, H+) plane. The polar angle standard deviation may measure the standard deviation in the patterns of approach and avoidance behavior. It may represent the spread of positive and negative preferences across the categories of evaluation items and may be a measure of the breadth of the user’s portfolio of preference.

3) A mean radial distance or reward-aversion consistency (r) value 1006, which is the average (mean) Euclidian distance of the data points in the (H₋, H+) curve to the origin 705. The mean radial distance defines how the user 128 can have strong positive and negative preferences, i.e., biases, for the same thing, reflecting conflict, or have low positive and negative preferences for something, reflecting indifference.

4) A radial distance standard deviation or consistency range (σr) value 1008, which is the standard deviation of the radial distances of the points in the (H₋, H+) plane to the origin 705. The radial distance standard deviation reflects how much the user 128 goes between having conflicting preferences and having indifferent preferences.

In some embodiments, the plotting tool 116 may be configured to generate all three plots: trade-off, value function, and limit function from the generated relative preference data. An evaluation of all three plots provides significant information for predicting a diagnosis. Nonetheless, it should be understood that in other embodiments the plotting tool 116 may be configured to generate only one of the trade-off, value function, or limit function plots. In other embodiments, the plotting tool 116 may be configured to generate some combination of the trade-off function, value function, or limit function plots that is less than all three plots.

The demographic data analyzer 122 may analyze the user demographic data 130 and extract one or more demographic features therefrom as indicated at step 234. Optionally, e.g., in some embodiments, the social data analyzer 124 may analyze the user social data 138 and extract one or more social features therefrom as indicated at step 236 (FIG. 2E). Optionally, e.g., in some embodiments, the historical purchase data analyzer 126 may analyze the user historical purchase data 140 and extract one or more historical purchase features therefrom as indicated at step 238.

The profile generator 104 may construct the user profile 144 as indicated at step 240. The user profile 144 may include one or more of the values 802-810, 902-912, and 1002-1008 derived from one or more of the plots 500, 600, and 700 in the user profile 144 as the judgment variables 148. The user profile 144 also may include the one or more demographic features 150 extracted from the user demographic data 130. In some embodiments, the user profile 144 also may include the one or more social features 152 extracted from the user social data 138 and/or the one or more historical purchase features 154 extracted from the user historical purchase data 140.

The profile generator 104 may input the user profile 144 to the ML model/classifier 106, as indicated at step 242. The ML model/classifier 106 may generate the prediction or classification 144 for the user 128 based on the user profile 144, as indicated at step 244. Processing may then be complete as indicated at Done step 246.

### ML Model/Classifier

As described, in some embodiments, the MLmodel/classifier 106 may be a random forest model or a gaussian mixture model.

### Random Forest

A random forest model is a machine learning method for classifying objects based on the outputs from a plurality of decision trees, usually a large number of trees. A trained random forest model may output a classification based on the votes of the plurality of decision trees based on the input to the trained random forest model. In some embodiments, each decision tree may include one vote, while in other embodiments, the decision trees may have different numbers of votes. Each decision tree of a random forest model may be trained on a bootstrap sample of available data, and each node in a decision tree can be split by one or more variables. The process of constructing a Random Forest model may include: generate a number of bootstrap sample sets; grow a decision tree for each bootstrap sample set; during the growing, randomly select a number of variables at each potential split (random feature projection); and combine the decision trees to form the Random Forest model. For regression, the output may be the average output among all the decision trees. For classification, the output may be determined by considering the vote of all the decision trees.

Depending on the size of the initial training data set, a random forest model can be trained to produce highly accurate classifications; handle large number of input variables; estimate the importance of variables for classification; estimate missing data; maintain accuracy when a large portion of data is missing; facilitate computing proximity of data classes for detecting outliers and for visualizing the data; and facilitate experimental detection of interaction of variables. Random Forest can both provide automatic variable selection and describe non-linear interactions between the selected variables.

In some embodiments, one or more of the fifteen judgment variables may be used as a main set of features for a broad array of machine learning techniques including Random Forest (RF) and balanced Random Forest (bRF). For a predicting a neurological condition or diagnosis, predicting consumer behavior, or predicting financial risk or behavior, the target variable, which represents the variable being predicted by the ML model/classifier 106, may be divided into high and low classes based on thresholds with clinical utility (i.e., yes/no for illness) or the utility from a design and service provision. All values below the threshold may be labelled as ‘low’ and values above and equal to the threshold may be labelled as ‘high’.

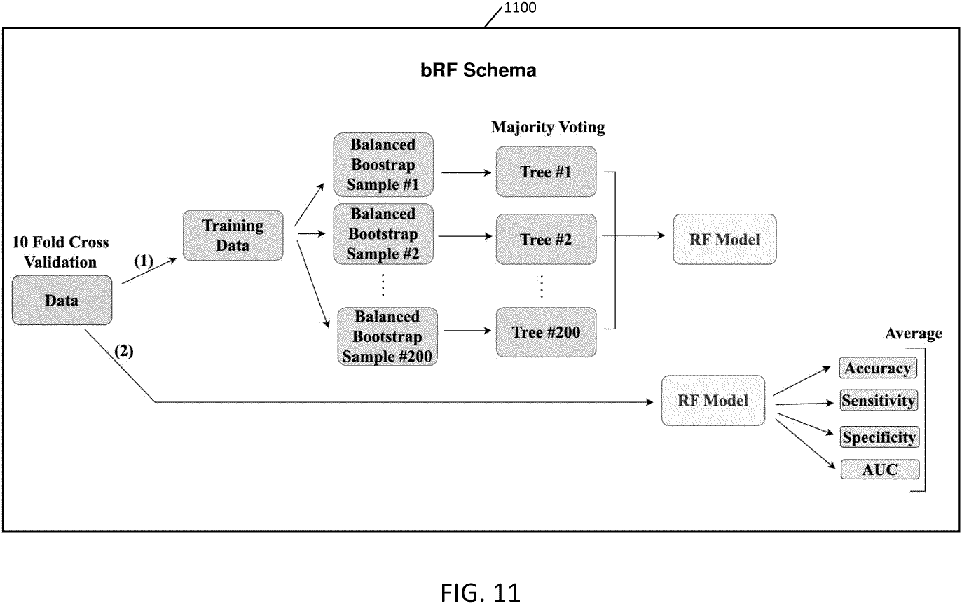

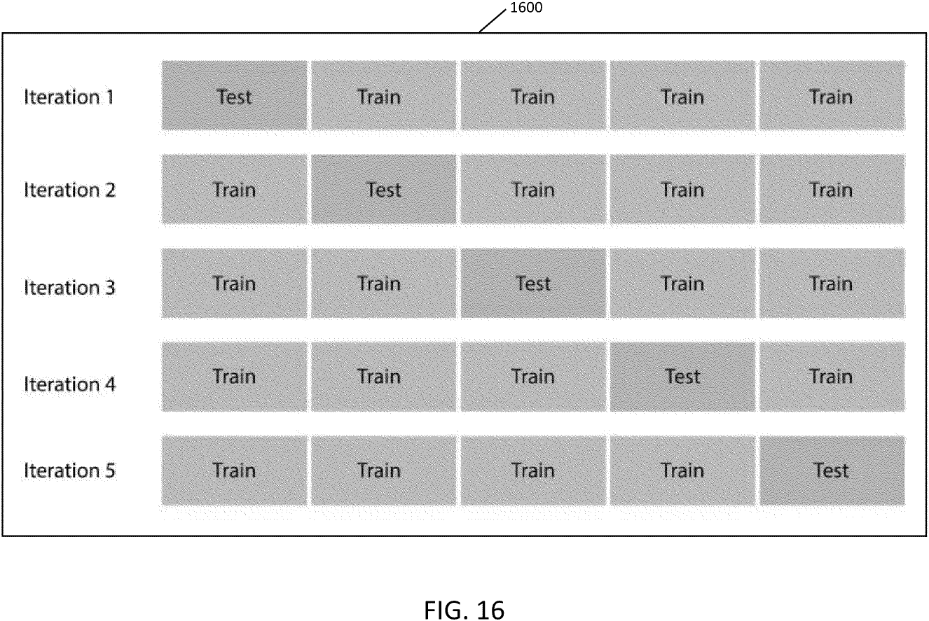

FIG. 11 is a highly schematic illustration of a general, example schema 1100 for an RF and/or bRF-based ML model/classifier in accordance with one or more embodiments. The ML model/classifier may be evaluated by using N-Repeated Stratified K-fold Cross-Validation to validate the performance of models. The dataset may be divided into K-folds in a stratified way, e.g., keeping the original ration of the classes in each split. Then, in each iteration, one part of the dataset becomes the test set. FIG. 16 is a schematic illustration of a plurality of iterations 1600 of a ML model/classifier in accordance with one or more embodiments. This procedure may be repeated for N times, thus acquiring model validation metrics, e.g., accuracy, sensitivity, specificity, etc., that may be calculated by exploiting at the greatest extent the inherent information of the dataset.

Particularly useful for assessing the quality of a machine learning model, bootstrapping is a method of inferring results for a population from results found on a collection of smaller random samples of the population, using replacement during the sampling process.

For the training sample, an RF may boot-strap the sample so that the target variable, e.g., presence of SCD, history of depression, financial risk in the form of negative events, consumption variables, being predicted has a similar distribution to the total sample in each training trial, producing a unique decision tree. For a bRF approach, a random down-sampling of the majority class may be performed so that the majority and minority classes in the dataset are equal. Both RF and bRF approaches may be implemented for each threshold value of the target variable (e.g., a score of 1 vs. all other episodes of depression, or 1 or 2 episodes of prior depression vs. 3 or more episodes, etc.) bRF approach can be used in addition to the standard RF analysis because of the tendency for greater class imbalance target variable distributions to drive overfitting or reduce the sensitivity/recall of a result.

For both RF and bRF approaches, N trees within a forest may be produced. In FIG. 11, N=200. The decision trees may recursively split features relative to their target variable’s purity, and underlie a prediction’s precision. RF analyses are thus designed to find the optimal point that a predictive feature splits one dataset into two, so that both groups are more homogenized than the parent group. One measure of the target variable’s purity is the Gini importance, which quantifies how often a randomly chosen element from the parent set would be incorrectly labeled. When the training set for the current tree is drawn by sampling with replacement, about one-third of the cases may be left out of the sample. This out-of-bag (OOB) data may be is used to get a running unbiased estimate of the classification error as the decision trees are added to the forest. It may also be used to get estimates of target variable importance. After each decision tree is built, all of the data may be run down the tree, and proximities may be computed for each pair of cases. If two cases occupy the same terminal node, their proximity may be increased by one. At the end of the run, the proximities may be normalized by dividing by the number of decision trees. Proximities may be used in replacing missing data, locating outliers, and producing illuminating low-dimensional views of the data.

The RF/bRF model may then be assessed on the data that was set aside for testing. From this testing assessment, measures of accuracy, sensitivity, specificity, ROC AUC and balanced accuracy (mean of sensitivity and specificity) may be computed. The percentage of entire dataset in each class (based on the threshold values) is also important to assess against these metrics and determine the potential for overfitting. This entire procedure may be repeated for each threshold value of the target variable, e.g., low threshold for SCD vs. moderate threshold or high threshold on surveys that access SCD-related symptoms.

For insight into what drives the result of the RF/bRF model, the judgement and contextual variables can be sorted based on mean decrease in Gini scores and plotted in decreasing feature importance with the most important features listed on the top of FIG. 11. The higher the mean decrease Gini score, the more important the feature is for classification. The relative importance of the features may be analyzed by the normalizing the Gini score of each feature with the sum of all Gini scores. This may give the relative proportion of importance of each feature towards classification out of the entire feature set.

After the RF/bRF model is developed with training, and calibrated with accuracy metrics from testing, it can be used for classifying individuals with a likelihood score. Individual classification may be done by using the tested model with a target subject going through each branch of the decision tree to determine if they are classified as being “high” or “low” for a classification criterion. The best metric to use for reporting the likelihood of the prediction is the value returned by the “predict_proba(X)” function in sklearn in the Python language, which predicts class probabilities for each subject X. The predicted class probabilities of an input sample are computed as the mean predicted class probabilities of the decision trees in the forest. The class probability of a single decision tree is the fraction of samples of the same class in a leaf. This computation returns the class probabilities of the input sample(s) (i.e., person X). There are variants to such approaches such as the prediction of class log-probabilities for X. In this case, the predicted class log-probabilities of an input sample can be computed as the log of the mean predicted class probabilities of the trees in the forest. This type of approach also returns the class probabilities of the input sample(s). While the above description is given for the bRF, it may generalized. For example, the predict_proba() can be applied on any of the other classifiers, such as models involving logistic regression, support vector machines, decision tree classifiers, e.g., random forest (RF), Gaussian Process (GP), and graph neural net techniques.

### Gaussian Mixture Model

A Gaussian Mixture Model (GMM) is a clustering method based upon linear learning models. Clustering refers to an unsupervised machine learning technique that aims to group similar entities into clusters. A cluster is defined as a subset of similar objects, defined by certain parameters (means, covariances and mixing coefficients), within a larger set. A GMM is a probabilistic model that assumes all the data points are generated from a mixture of a finite number of Gaussian distributions with unknown parameters. The goal for a GMM is to maximize the likelihood function with respect to the parameters. The number of clusters specifies the number of components in the GMM. GMMs can be used to represent normally distributed subpopulations within an overall population. GMMs in general don’t require knowing which subpopulation a data point belongs to, allowing the model to learn the subpopulations automatically. A GMM attempts to find a mixture of multi-dimensional Gaussian probability distributions that best model the input dataset. GMM clustering can accommodate clusters that have different sizes and correlation structures within them.

For example, given a set of training data XL×M, where L is the dimension of the data and M is the number of samples, a GMM learns K centroids such that each sample can be assigned to the closest centroid. Suppose the observed feature vectors form a feature space and the appropriate K centroids in the high-dimensional feature space are known. For example, a pipeline defines a function f: RL→RK that maps the observed L-dimensional feature vector to a K-dimensional feature vector (K<L). For instance, the affiliations for each observed feature vector (with respect to the K centroids) may first be calculated and then the affiliations can be used as morphological signatures to represent each key point in the feature space.

GMMs can perform either hard clustering or soft clustering on query data. With hard clustering, the GMM may assign query data points to the multivariate normal components that maximize the component posterior probability, given the data. That is, given a fitted GMM, the ML model/classifier 106 may assign query data to the component yielding the highest posterior probability. Hard clustering may assign a data point to exactly one cluster. In other embodiments, the ML model/classifier 106 can use a GMM to perform a more flexible clustering on data, referred to as soft (or fuzzy) clustering. Soft clustering methods assign a score to a data point for each cluster. The value of the score indicates the association strength of the data point to the cluster. As opposed to hard clustering methods, soft clustering methods are flexible because they can assign a data point to more than one cluster. When the ML model/classifier 106 performs GMM clustering, the score may be the posterior probability.

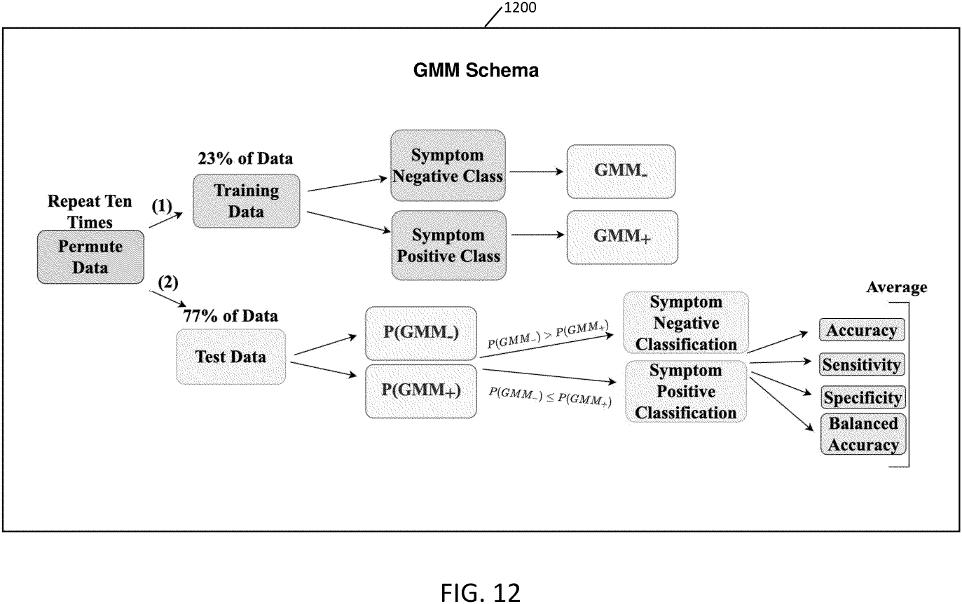

FIG. 12 is a highly schematic illustration of a general, example schema 1200 for a GMM-based ML model/classifier in accordance with one or more embodiments.

As described, GMMs contain a mixture of different gaussian distributions, each having a corresponding probability density. Sampling from a GMM is equivalent to randomly choosing one of the gaussian distributions in the GMM in accordance with its corresponding probability, and subsequently drawing a sample from that specific gaussian distribution that was chosen. GMMs are useful because, by varying the number of gaussian distributions in the GMM, probability density functions of a wide variety of distributions can be approximated.

Referring to FIG. 12, a suitable procedure for the GMM classification approach may be as follows. First, using the training data, the preference features may be segregated into two separate groups, based off their corresponding labels, e.g., either yes/no for illness. Then, each of the two groups of preference variables may be fit to the GMM, resulting in two GMMs. The number of mixtures may be chosen for each GMM to be between one and ten, by choosing the number of mixtures that minimizes the Bayesian Information Criteria (BIC) score. Generally, lower BIC scores correspond to better model fits of data. As a result, there are two GMMs corresponding to each label., e.g., either yes/no for illness.

For a point to be classified, e.g., based off of one or more judgment variables, the density of that point for the positive class GMM and the negative class GMM may be computed. If the probability of the symptom negative GMM of that point is larger than the probability of the symptom positive GMM, then the point may be classified as being symptom negative. Otherwise, the point may be chosen to be symptom positive. The metrics that may be used to evaluate the testing dataset may be accuracy, sensitivity, specificity, and balanced accuracy. This process may be repeated, e.g., ten times, by choosing a random, e.g., 77/23, train/test split of the data in each iteration. The accuracy, sensitivity, specificity, and balanced accuracy may be averaged over these ten iterations. This is analogous to cross-validation or bootstrap. Furthermore, other boot-strapped statistics may be obtained if desired, such as the variance of the accuracy, sensitivity, specificity, and balanced accuracy, or higher moments of the statistics as well.

After a GMM model is trained, the mean values and variances of each of the judgement features can be compared between the GMM for the positive class and GMM for the negative class. The mean can be computed by adding up the means multiplied by the corresponding occurrence probabilities of each gaussian in the mixture in the GMM. The variance can be computed by adding up the variances multiplied by the corresponding occurrence probabilities of each gaussian in the mixture for each GMM. This process may assumes that the gaussians in the GMM are independent of each other. Comparing the mean and variances of the judgment features in each group may yield an understanding of how judgement variables’ values differ based off their respective symptom categories.

Suitable tools for creating and operating GMMs include the Statistics and Machine Learning Toolbox from The MathWorks, Inc.

### Predicting Neurological Disease/Condition

Pilot data testing has indicated a quantitative approach for objective detection of Subjective Cognitive Decline (SCD) in a diverse population with and without demographic vulnerabilities. A multi-variable regression (MVLR) without training-test sets was run to determine model accuracy to discriminate SCD. Multivariable regression models are used to establish the relationship between a dependent variable (i.e. an outcome of interest) and more than 1 independent variable. This was followed with Machine Learning (ML) analysis using Gaussian Process (GP) classification for prediction accuracy, sensitivity, and specificity. Suitable tools include the Stata statistical software from StataCorp LLC and the R software environment for statistical computing and graphics from the R Foundation. Statistical significance may be established if p-value < 0.05.

For assessing Subjective Cognitive Decline (SCD) from MVLR analyses, Loss Resilience (LR) alone showed slightly better model accuracy than Insurance as illustrated in Table 1 below. These were compared to approach or positive preference variables such as the reward tipping point or Reward TP. When age, income, and education (edu) were included in the LR model, the accuracy increased to 97%. In contrast, when age, income, and education were added to the Insurance model, the accuracy only increased to 86%. It should be noted that putting LR and Insurance together with age, income, and education changed effects in a major way (98%).

TABLE 1

| Judgment Variables/Demographic Information | Average Diagnosis Prediction Accuracy by trained ML Model/Classifier (%) |
|---|---|
| Reward Tipping Point | 55.68 |
| LR | 61.05 |
| Insurance | 55.96 |
| LR, age, income, education | 96.63 |
| Insurance, age, income, education | 85.50 |
| LR, Insurance, age, income, education | 97.56 |

These analyses were followed with ML-based prediction using the following settings to train the Gaussian Process (GP) classifier. A radial basis function kernel was used, and the prediction accuracy was averaged over 10 cross-validations. The variance of the accuracies for all reported measurements was less than one percent with ten iterations. For each train/test split, data were chosen with a random number generator so that 66% was training data and 33% was testing data. Using the LR judgment variable, alone, the model was 94% accurate to predict SCD, and resampling confirmed there was no model overfitting. This accuracy increased to 95% when age, income, and education demographic features were added to the model, and remained at 95% with the addition of the Insurance judgment variable. These ML results are comparable to MVLR results, demonstrating the consistency of this approach using informative judgment variables to predict SCD with high accuracy.

Predictive ML approaches may use logistic regression (LR), support vector machines (SVM), decision tree classifiers [e.g., random forest (RF)], Gaussian Process (GP) and graph neural net techniques. In this example, GP was used. The models may be trained and validated on a training dataset, and the performance may be evaluated on a testing set. For all ML models, boot-strapped accuracy, sensitivity, and specificity may be reported. Due to data imbalance, these measures should be interpreted with respect to the size of the minority class (i.e., SCD positive). Features may be selected based success with MVLR preliminary testing. Following feature selection, the robustness of the selected features in the different models may be tested. In this example, GP was used. Features used as input variables start with the 15 judgment, e.g., preference, variables. This may be extended to include moments and fitting parameters if preference variables, alone, do not produce non-trivial predictive power. Demographic features of interest may include age, income, and education, but can be extended if further associative analysis suggests or requires it. Where longitudinal data is available, autoregressive GP techniques can be used and the inducing points method if scalability is of importance. Kernel design for GPs may be implemented through visualization of the features as well as evaluation of their covariances.

The specific problem of predicting a neurological condition in a subject, such as SCD, e.g., to allow for early intervention and care, is one instantiation of a larger construct, whereby the present disclosure can predict mental conditions, such as a history of depression, or predict risky behavior and negative events around financial judgments or predict commercial judgments and design products and services around them (e.g., types of cars, price points, and services). Use of variables reflecting biases in judgment, along with demographic and other contextual variables that affect judgment (e.g., age, education, past history of negative financial events) allows use of a wide array of machine learning approaches (e.g., random forests, support vector machines, neural nets, Gaussian mixture models) to predict neurological and mental health conditions, and conditions related to high risk financial judgments or biases in commercial judgments that can be used to determine what products are designed, how they are priced, and a broad array of supply chain issues regarding the supply of a product or service due to consumer biases.

The present disclosure represents a significant increase in processor and memory efficiency as compared to many current machine learning approaches, such as deep learning networks. For example, many deep learning networks include tens or hundreds of hidden layers resulting in large numbers of weights. Such networks require large memory resources. For example, the well-known AlexNet Convolutional Neural Network (CNN), which can classify images to 1000 categories, has 230 million parameters and performs one and a half billion operations to classify one image of size 227×227×3. Loading and running such massive networks often requires significant memory resources and specialized processor resources, such as Graphical Processing Units (GPUs) and/or Tensor Processor Units (TPUs). The present disclosure includes up to fifteen judgment variables, and may involve less than 100 total judgment and demographic or survey variables, which is orders of magnitude fewer than the parameters required by many current deep neural networks. Accordingly, the present disclosure provides significant memory savings. The present disclosure also executes faster and does not require GPUs and/or TPUs as compared to many deep neural networks.

The present disclosure can also generate accurate predictions of conditions or diseases without the subject having to meet with and/or be examined or evaluated by clinicians or other professionals or undergo medical testing. For example, the rating app may be loaded on a subject’s smartphone, allowing the subject to generate rating data anywhere. The present disclosure may thus result in significant cost savings.

### Predicting Consumer Behavior

Determining what a consumer wants to buy and what they are willing to pay for a particular product, such as a car model, is a critical for designing products, services and the fee structure a market will support. Accurately predicting customer behavior, such as whether a new product will be successful, whether a product is priced correctly, whether a given consumer will make a particular purchase and, if so, how much they are willing to pay, however, is extremely difficult. Companies use many different techniques to try and predict customer behavior, such as focus groups, test marketing, polls, interviews, and market research, among others. These techniques, however, can be time-consuming and expensive. They can also fail to accurately predict consumer behavior. Many big data approaches that try and characterize consumer behavior by collecting as much data as possible for predictions based on behavioral tracking often have very high p-values but very small effects, e.g., often in the range of 1% to 5% without the prediction accuracies above 80% as with the present disclosure. Predicting what consumers will acquire is fundamental for supply chain management of available stock, for designing changes to existing products to meet demand, for determining what services consumers will need for potential acquisitions. The prediction of supply needs and relative apportionment of production between potential product models, however, depends just on what was sold in previous months/years or what focus groups and non-quantitative surveys suggest. These existing frameworks for predicting consumer behavior produce low accuracies, producing significant problems for manufacturers and stores and for supply chain management.

The present disclosure is directed to low-cost, highly accurate systems and methods for predicting customer behavior.

### Predicting Car Cost and Model Acquired

Here is an example of developing a “scoring” method for first predicting judgment variables in one sample, and then computing them for a second unrelated sample. These “scored” judgment variables are then shown to fundamental for the prediction of what subjects are willing to pay for particular car models. In this example, scoring of two judgment variables was performed with one dataset using an RF approach (CRT modeling), and then used to reconstitute these two variables with a second dataset for which no judgment-based picture-rating task had been performed. These two judgment variables were then used in this second dataset for high accuracy prediction of payment willingness and car model preference with an RF approach.

The first sample involved 3476 subjects interviewed for the scoring of RA and LR judgment variables, whereas the second dataset involved 17,430 participants, of which over 4600 subjects had car preference and acquisition data, and were used for ML prediction analyses. Subjects had reported acquisition and price paid for cars, hybrid vehicles, minivans, trucks, SUVs, and electric vehicles. Variables used as input variables included the judgment variables of RA and LR, marital status, age, income, gender, education, and metropolitan proximity. A CRT-based Random Forest (RF) approach was used with 70% of the subjects used for training, and 30% used for testing; cross-validation with 100 folds was also used, to produce an overall accuracy of 90.0%.

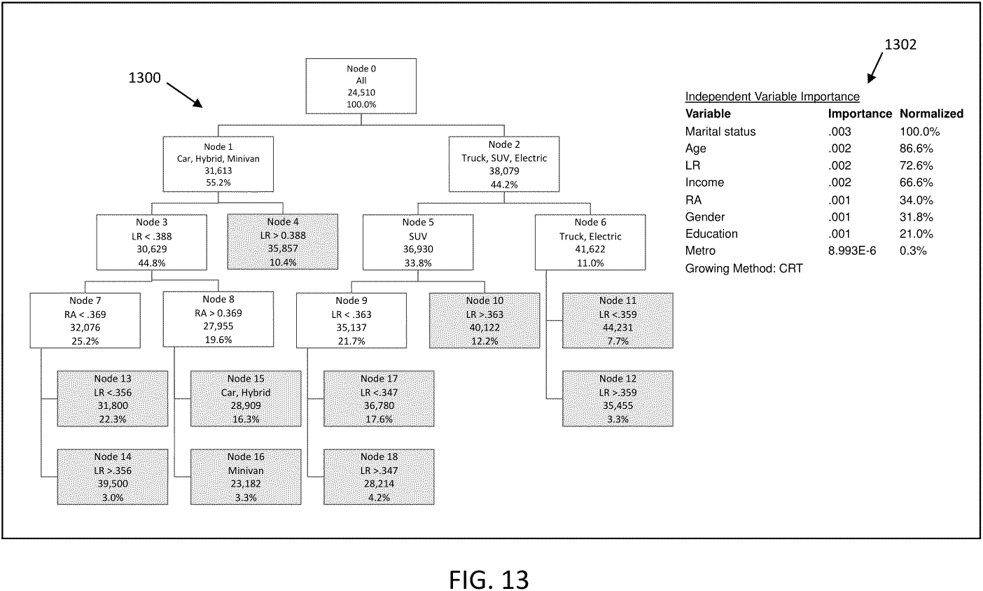

FIG. 13 is an illustration of a pruned example of the recurrent partitioning 1300 and a tabulation of variable importance 1302 using Gini scores in accordance with one or more embodiments.

Notable is the segmentation of listed car price and models by Risk Aversion (RA) and Loss Resilience (LR) variables.

### Fashion Purchase Choice Prediction

Another example of determining what a consumer wants, by what they are willing to pay for specific clothing fashions, is critical for designing products, services and the fee structures a market will support. In the framework of women’s fashion, we again used a “scoring” method for first predicting judgment variables in one sample where a picture rating task had been performed, and then computed them for a second unrelated sample where a picture rating task had not been performed. Using classification regression tree (Random Forest, RF) rules determined from the first sample, both RA (59 nodes) and LR (67 nodes) variables were estimated for the second sample using gender, age, education and ethnicity as common variables between the two unrelated samples. In the initial sample with the rating task, mean RA across the sample was 0.420, and in the second sample which did not differ epidemiologically from the first, RA was computed to be 0.419. In the initial sample with the rating task, mean LR across the sample was 0.392, and in the second sample which did not differ epidemiologically from the first, LR was computed to be 0.390.

These “scored” judgment variables are then shown to be fundamental for the prediction of what subjects are willing to pay and for which types of clothing fashions. We specifically used the scored judgment variables plus demographics and consumption information to show that out of 10 terminal nodes in a pruned Random Forest (RF) analysis, that 8 of 10 terminal nodes were directly driven by RA or LR, and two terminal nodes were secondarily driven by one of these judgment variables.

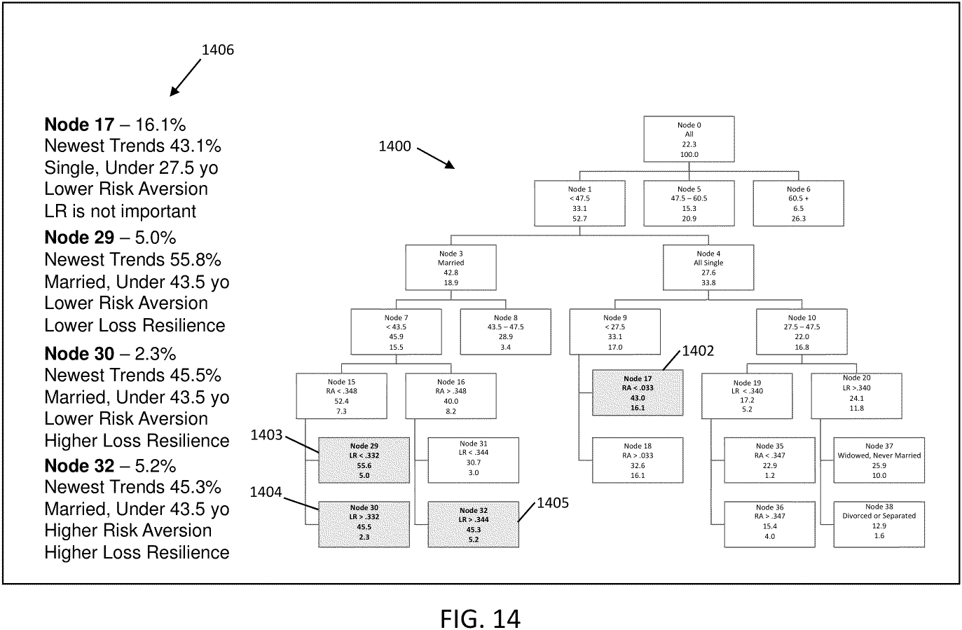

FIG. 14 is a schematic illustration of an example of a pruned decision tree 1400 with 10x K-fold validation for predicting new trend fashion preference of a random forest classifier in accordance with one or more embodiments. Four terminal nodes 1402-1405, e.g., Node 17, Node 29, Node 30, and Node 32, which are highlighted, show relative weights for “Newest Trends” above 40%. A legend 1406 provides details on the nodes 1402-1405. Nodes 1403-1405 (Nodes 29, 30, and 32) relate to married women under 43.5 years old (yo). These nodes have distinct profiles for RA and LR. Node 1402 represented single women under 27.5 years old (yo) for whom only RA was important.

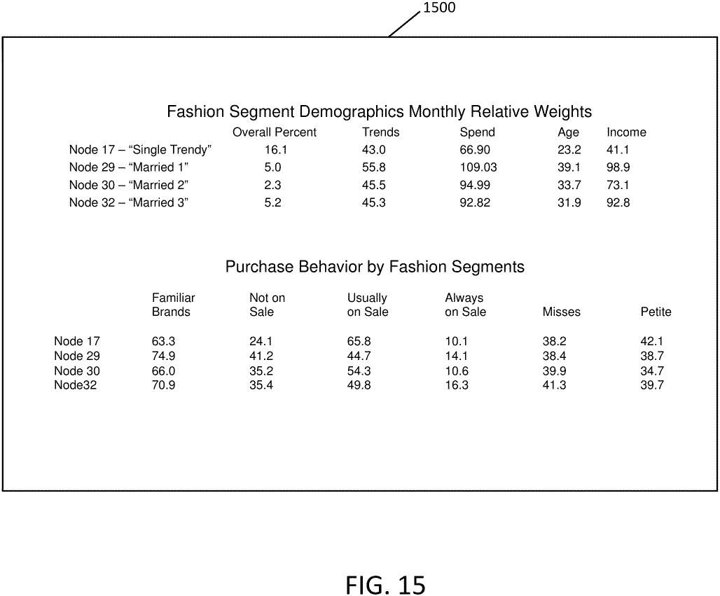

FIG. 15 is an illustration of the profiles 1500 of the tested subjects for nodes 1402-1405 (Nodes 17, 29, 30, and 32) in accordance with one or more embodiments. The profiles indicate the subjects available income (yearly in thousands) and amount spent on “newest trend” clothing (weighted amount per month). The nodes 1402-1405 (Nodes 17, 29, 30, and 32) of subjects further differed by their purchase behavior in terms of buying familiar brands, items not on sale, items usually on sale, items always on sale, and items that were Misses or Petite sizes. This type of prediction may be a core necessity for market segmentation, and is fundamental for designing products, services and the fee structures the market will support for women’s fashion.

### Predicting Financial Behavior

Predicting the financial risk presented by a prospective borrower, such as the likelihood of default, or other financial behaviors, such as a person’s financial risk tolerance, is difficult and error-prone. Lending institutions, for example, employ sophisticated underwriting departments that analyze a prospective borrower’s credit history. Brokerage houses may rely on surveys to determine a person’s risk profile, such as the 14 FICO questions. FICO was founded in 1956 as Fair, Isaac and Company and sold its first credit scoring system two years after the company’s creation. FICO went public in 1986 and is traded on the New York Stock Exchange. The company debuted its first general-purpose FICO score in 1989. FICO scores are based on credit reports and “base” FICO scores range from 300 to 850, while industry-specific scores range from 250 to 900. Lenders use the scores to gauge a potential borrower’s creditworthiness. Fannie Mae and Freddie Mac first began using FICO scores to help determine which American consumers qualified for mortgages bought and sold by the companies in 1995.

Today, the traditional sources of funds, such as private capital, banks, and securitization, have issues, such as regulatory, heightened credit cycle concerns, etc. There are also borrower issues where consumer loans tend to enter default in down cycles. There are also systemic issues that reduce the quality of underwriting, such as lower standards due to online lending competition and limitations of automated credit checks. The present disclosure including combining judgment variables with machine learning (a form of explainable artificial intelligence or xAI) can be advantageous in the field of ‘automated’ credit check issue.

The present disclosure including the use of judgment variables representing biases in judgment with machine learning (ML) for assessing financial risk presents a significant advantage and solution to current problems. Use of judgment variables with contextual variables (e.g., demographics, survey material on activities) as input to machine learning allows high accuracy prediction of risk around financial choices and negative events.

### Segmenting Financial Risk

Determining what financial risk is associated with individuals, characterized by behavior and demographics, is critical for determining the loans they might receive from individuals or their peers (i.e., peer to peer lending), the terms of financial agreements, or the frameworks put in place for recuperation of loans made but not readily recovered. With data from 4105 subjects, systems and methods can segment financial risk using judgment variables, demographics, and questions from the fourteen questions asked for FICO assessments. In this cohort, subjects may perform a picture rating task for computing the judgment variables as described, along with responding to the FICO questions and demographic questions. Random Forest (RF) analysis with 10x cross validation may be used to show distinct segments of risk associated with negative credit events.

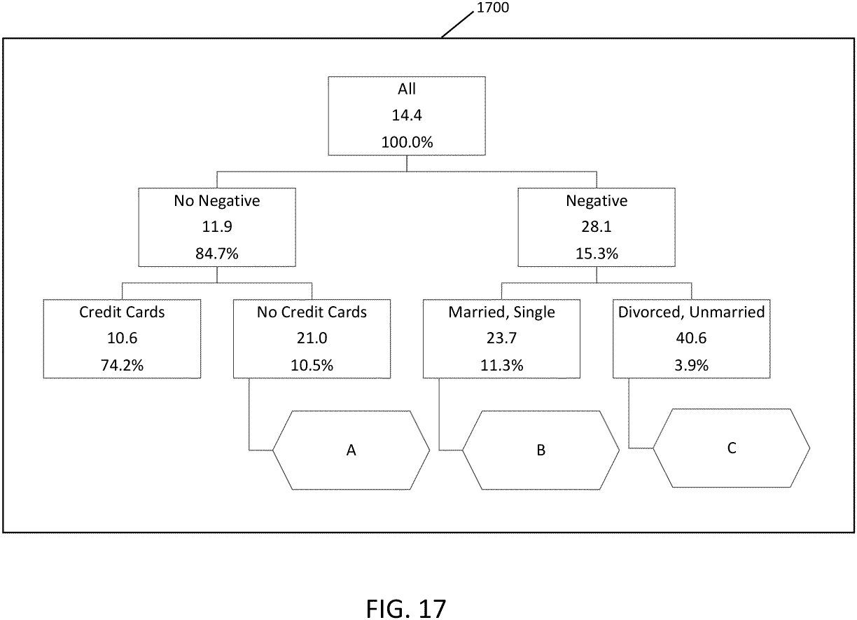

FIG. 17 is a schematic illustration of an example Random Forest analysis 1700 with two major segments for negative credit events in accordance with one or more embodiments. The RF analysis 1700 overall represented just 15.3% of the total sample of 4105 subjects.

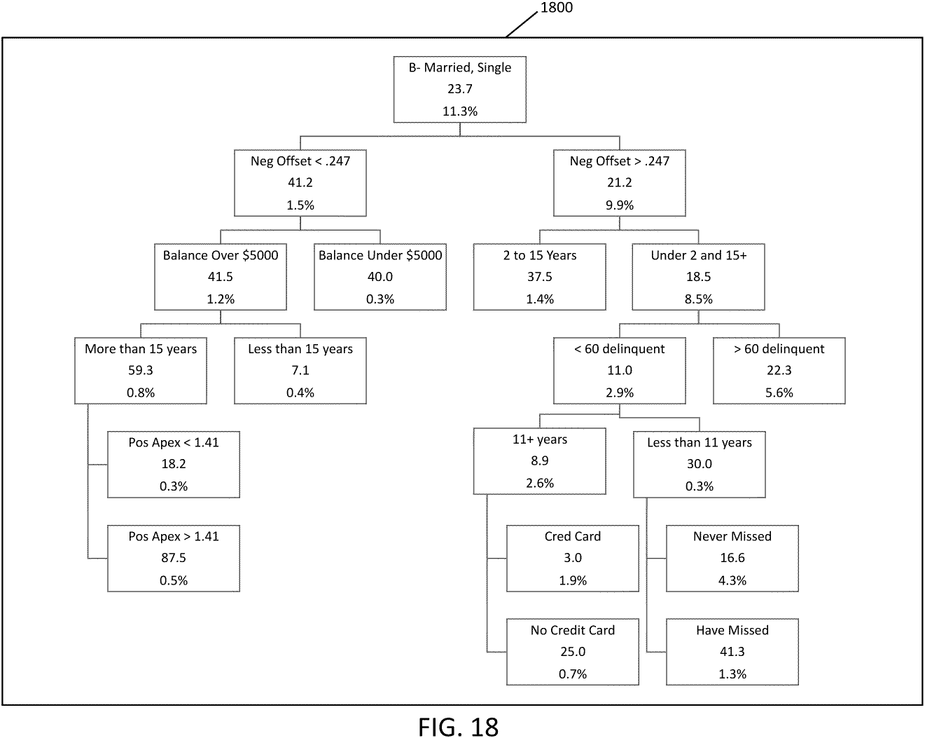

FIG. 18 is a schematic illustration of an example of segment B 1800 from the RF analysis 1700 of FIG. 17 in accordance with one or more embodiments. As shown in FIG. 18, married and single individuals (vs. divorced or unmarried) represented 11.3% of the total sample and two judgment variables were critical for the recurrent segmentation of the tree. Specifically, the judgment variable of Insurance and Peak Positive Risk framed primary initial partitionings and terminal partitionings of the data, respectively. As shown, variables such as being married or single, Insurance value, balance due, years with credit cards, Peak Positive Risk, number of days delinquent, history of having a credit card may be aggregated and evaluated to predict the likelihood an individual is to have negative risk outcomes. In this particular RF analysis, having an Insurance value of more than 0.247 characterized 9.9% of the subjects with credit problems, indicating they would likely need to have terms helping to protect against bad credit events, e.g., limited amounts loaned, longer term paybacks, greater collateral, etc.

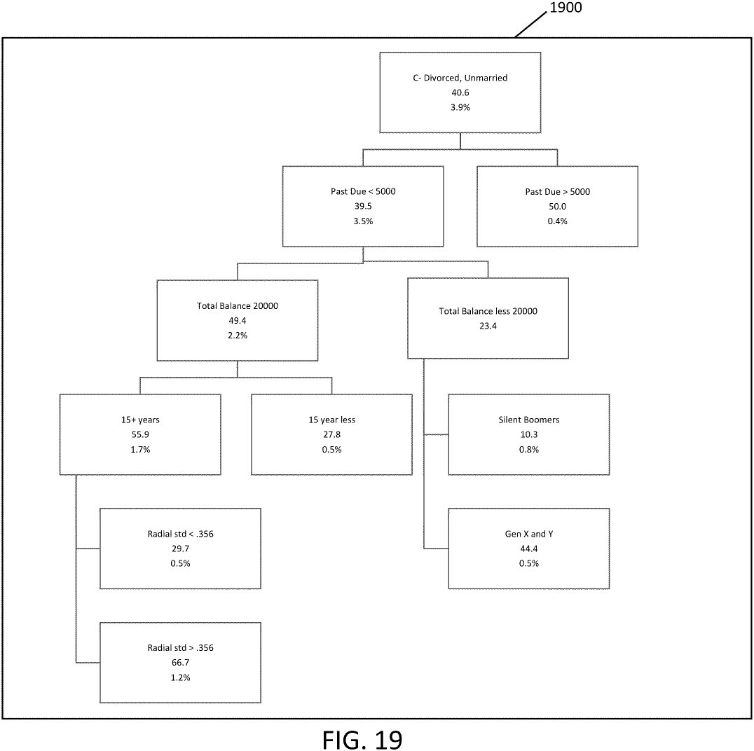

FIG. 19 is a schematic illustration of an example of segment C 1900 from the RF analysis 1700 of FIG. 17 in accordance with one or more embodiments. As shown in FIG. 19, in the case of those who are divorced or unmarried, the judgment variable of consistency range defined two terminal nodes that characterized 1.7% of the total 3.9% of negative events related to individuals who are divorced or unmarried. This emphasizes that three distinct judgment variables are significant for segmenting groups of subjects who ended up with negative credit events, but for very different reasons. Such risk segmentation based at least in part on rating data is an improvement to current techniques for predicting credit risk.

### Predicting Responses to FICO Questions

Predicting specific financial risks as defined by the FICO questions has been a significant challenge, especially when standard credit behavior is not available, as for the tens of millions of individuals in the USA with no credit history (i.e., the “unbanked”). Unbanked individuals include students who often must have parental co-signatures to start using credit.

The present disclosure can generate predictions with greater than 60% accuracy of subjects’ answers to nine of the fourteen FICO questions. That is, judgment behavior alone can predict with greater than 60% accuracy nine of the fourteen FICO questions. This type of information is critical for determining the loans individuals might receive, or terms for peer to peer lending, the terms of financial agreements, or the frameworks put in place for recuperation of loans made but not readily recovered.

In an example, 3476 subjects were assessed who had performed a picture rating task to allow judgment variable computation along with filling out the FICO questions. The fifteen judgment features were computed and a split sample (test-retest) framework was used where one cohort produced the ML model/classifier 106 and the second was tested against it for aggregating summary statistics for successful prediction. In this way, the most important judgment variables for making predictions were determined.

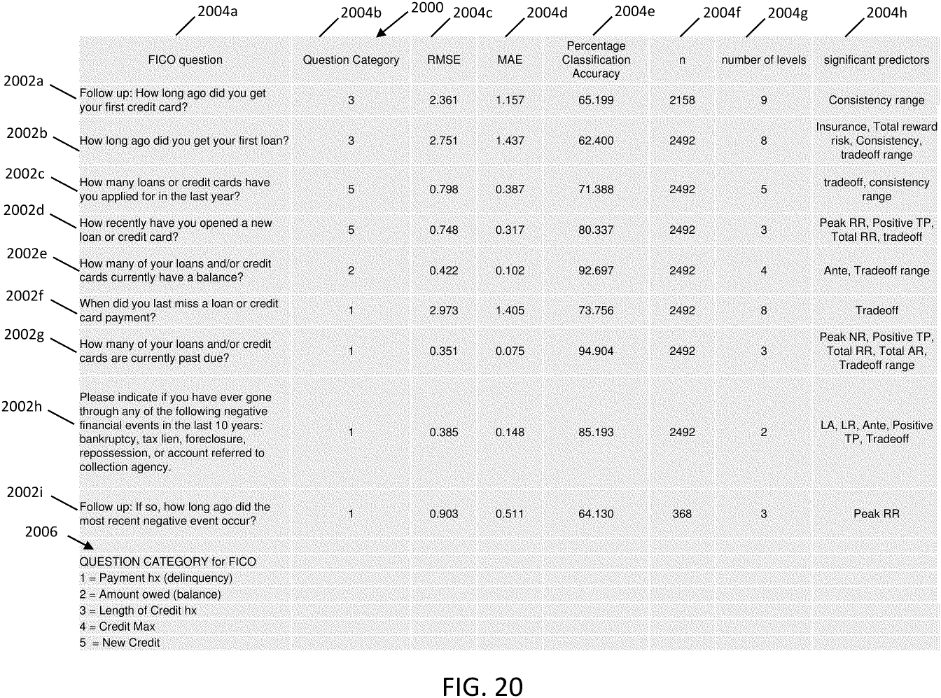

FIG. 20 is an illustration of example results 2000 in table form from MVLR analysis of a plurality of the FICO questions in accordance with one or more embodiments. The table 2000 may include a plurality of rows 2002a-i and columns 2004a-h defining cells or records. Each of the rows 2002a-i may contain data for a respective one of the FICO questions. Column 2004a may contain or other identify the FICO questions. The results table 2000 may present the results of the MVLR analysis for those FICO questions found to have classification accuracies above a predetermined threshold, e.g., 60%. As illustrated by the number of rows 2002, nine of the FICO questions had classification accuracies above the threshold. The FICO questions may be organized into categories. For example, the questions may be organized into five categories as indicated in a legend 2006 and column 2004b may identify the category of the FICO questions, e.g., category 1 may correspond to a category identified as ‘payment history (hx) (delinquency category)’. Column 2004c lists the Root Mean Square Error (RMSE), which may be one metric used by the data quality assurance engine 121 to assess regression outcomes. Lower RMSE values may indicate stronger prediction results. Column 2004d lists the Mean Absolute Error (MAE), which is the arithmetic mean of the absolute errors. MAE is another metric utilized by the data quality assurance engine 121 to assess how well a regression analysis performed. As with the RMSE, the lower the MAE value, the better the outcome. Column 2004e provides the percentage classification error based on the retest analysis of the initial (i.e., test) analysis with multivariate logistic regression. Column 2004f lists how many subjects (n) had data for the FICO question along with complete rating data 130 for judgment variable computation and input into the MVLR analysis to predict that target FICO variable. Column 2004g lists the number of discrete levels for response that were asked for each question, i.e., 2 meant a Yes/No, and any number of 3 or more meant there were three or more response options. Column 2004h lists the judgment variables that were primarily behind the output of the MVLR analysis.

As shown by the RSME, MAE and percentage classification accuracy columns 2004c, 2004d, and 2004e, an accuracy ranging from 64% to 94% was generated for questions in the credit delinquency category, category 1. The highest accuracy was generated for predicting how many loans were past due.

The FICO questions regarding credit history are widely used for credit rating and setting load amounts and loan terms. In this example, the ML prediction system 100 can accurately predict a subject’s responses to three-fifths of the FICO questions, including the majority of the delinquency questions, using judgment variables alone. When other variables beside those for judgment are included, these accuracies increased.

### Other Preference Collection/Computation Methods

It should be understood that the relative preference data may be computed from other information besides ratings of evaluation items 110.

### Demographic/Lifestyle/Consumption Dataset

In some embodiments, the profile generator 104 may utilize a “scoring” methodology between two datasets to compute relative preference data and graphs from which one or more of the judgment variables 148 may be computed. The profile generator 104 may first predict one or more judgment variables in one sample, e.g., where a picture rating task had been performed, incorporating a broad array of non-judgment variables, such as demographics or survey responses for this prediction of the judgment variables, and then computing the judgment variables in a second unrelated sample where a picture rating task had not been performed, but the other non-judgment variables had been collected.

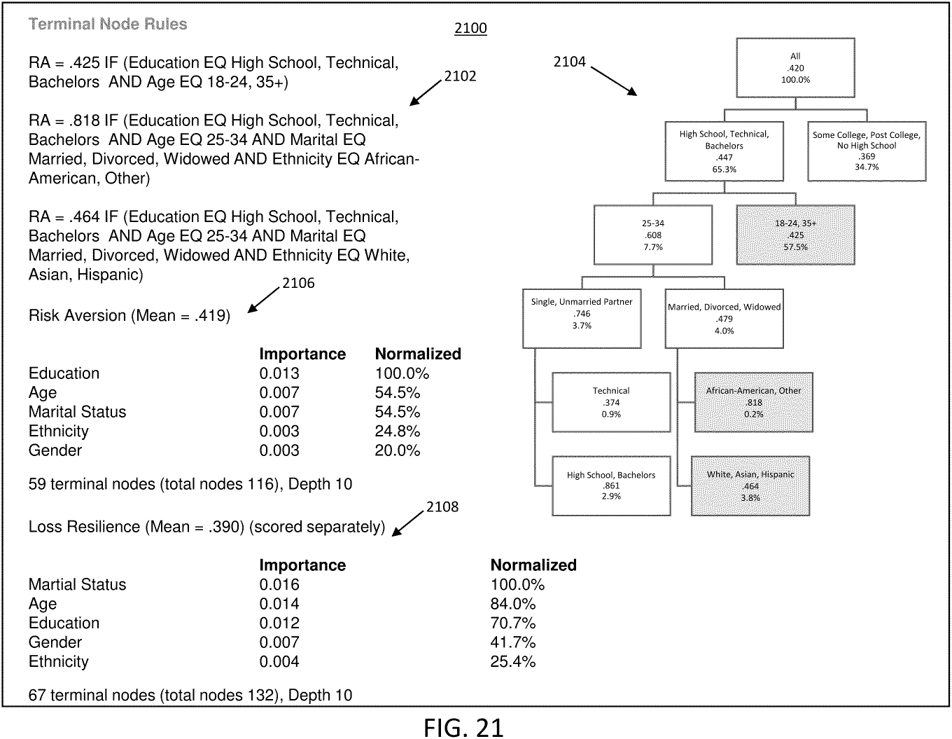

FIG. 21 is a schematic illustration of a scoring approach 2100 in accordance with one or more embodiments. The approach 2100 may include or use partitioning rules 2102 from a classification regression tree, e.g., Random Forest (RF), determined from a first sample, to estimate judgment variables for RA (59 nodes) and LR (67 nodes) in a second sample. The same gender, age, education and ethnicity variables were common variables between the two unrelated samples. In the initial sample with the rating task, mean RA across the sample was 0.420, and in the second sample which did not differ epidemiologically from the first, RA was computed to be 0.419 using the rules from the RF analysis done in the first sample, to reconstitute RA in the second sample. In the initial sample with the rating task, mean LR across the sample was 0.392, and in the second sample which did not differ epidemiologically from the first, LR was computed to be 0.390. A pruned tree 2104 predicting RA and LR in the first cohort had a total of 126 nodes, and depending on the constellation of non-judgment, e.g., demographic, variables, very different RA values may be observed as noted in the pruned tree 2104. Overall, this reverse computation of RA from an RF tree produced an average RA very similar to the average RA of the initial sample, and the same was the case for LR. The relative weightings of contribution to prediction of RA and LR was quite distinct as shown with a listing of the Gini scores for RA as indicated at 2106 and LR as indicated at 2108. For RA, the following order of relative contribution was observed: education > age > marital status > ethnicity > gender. For LR, the relative contributions of these variables was: marital status > age > education > gender > ethnicity.

The profile generate can implement this approach of reconstituting or “scoring” the judgment variables from one dataset to another when the same variables used to “score” the judgment variables are available in each dataset. The power of this approach is that highly predictive and interpretable judgment variables can be used across a broader set of cohorts and used for highly accurate prediction as noted elsewhere in the present disclosure, even if a picture rating task had not be performed with that particular dataset.

### Data Processing System

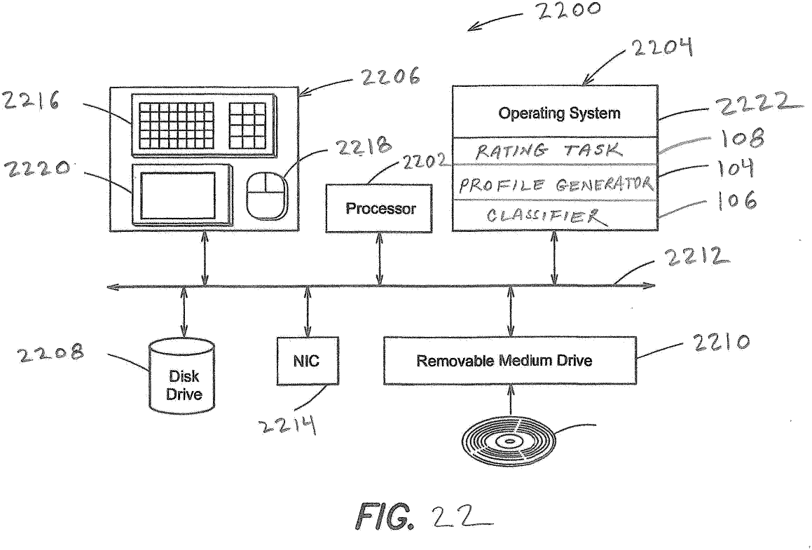

FIG. 22 is a schematic illustration of an example computer or data processing system 2200 for implementing one or more embodiments of the present disclosure in accordance with one or more embodiments. The computer system 2200 may include one or more processing elements, such as a processor 2202, a main memory 2204, user input/output (I/O) 2206, a persistent data storage unit, such as a disk drive 2208, and a removable medium drive 2210 that are interconnected by a system bus 2212. The computer system 2200 may also include a communication unit, such as a network interface card (NIC) 2214. The user I/O 2206 may include a keyboard 2216, a pointing device, such as a mouse 2218, and a display 2220. Other user I/O 2206 components include microphones, speakers, voice or speech command systems, touchpads and touchscreens, wands, styluses, printers, projectors, etc. Exemplary processors include single or multi-core Central Processing Units (CPUs), Graphics Processing Units (GPUs), Field Programmable Gate Arrays (FPGAs), Application Specific Integrated Circuits (ASICs), microprocessors, microcontrollers, etc.

The main memory 2204, which may be a Random Access Memory (RAM), may store a plurality of program libraries or modules, such as an operating system 2222, and one or more application programs that interface to the operating system 2222, such as the rating task 108, the profile generator 104, and/or the classifier 106.

The removable medium drive 2210 may accept and read a computer readable medium 2226, such as a CD, DVD, floppy disk, solid state drive, tape, flash memory or other non-transitory medium. The removable medium drive 2210 may also write to the computer readable medium 2226.

Suitable computer systems include personal computers (PCs), workstations, servers, laptops, tablets, palm computers, smart phones, electronic readers, and other portable computing devices, etc. Nonetheless, those skilled in the art will understand that the computer system 2200 of FIG. 22 is intended for illustrative purposes only, and that the present invention may be used with other computer, data processing, or computational systems or devices. The present invention may also be used in a computer network, e.g., client-server, architecture, or a public and/or private cloud computing arrangement. For example, the profile generator 104 and/or the classifier 106 may be hosted on one or more cloud servers or devices, and accessed by remote clients through a web portal or an application hosting system, such as the Remote Desktop Connection tool from Microsoft Corp.

Suitable operating systems 2222 include the Windows series of operating systems from Microsoft Corp. of Redmond, WA, the Android and Chrome OS operating systems from Google Inc. of Mountain View, CA, the Linux operating system, the MAC OS® series of operating systems from Apple Inc. of Cupertino, CA, and the UNIX® series of operating systems, among others. The operating system 2222 may provide services or functions for applications or modules, such as allocating memory, organizing data objects or files according to a file system, prioritizing requests, managing I/O, etc. The operating system 2222 may run on a virtual machine, which may be provided by the data processing system 2200.

As indicated above, a user, such as the user 128, may utilize one or more input devices, such as the keyboard 2216, the mouse 2218, and the display 2220 to operate the rating system 102, the profile generator 104, and/or the classifier 106.

### Distributed Computing Environment

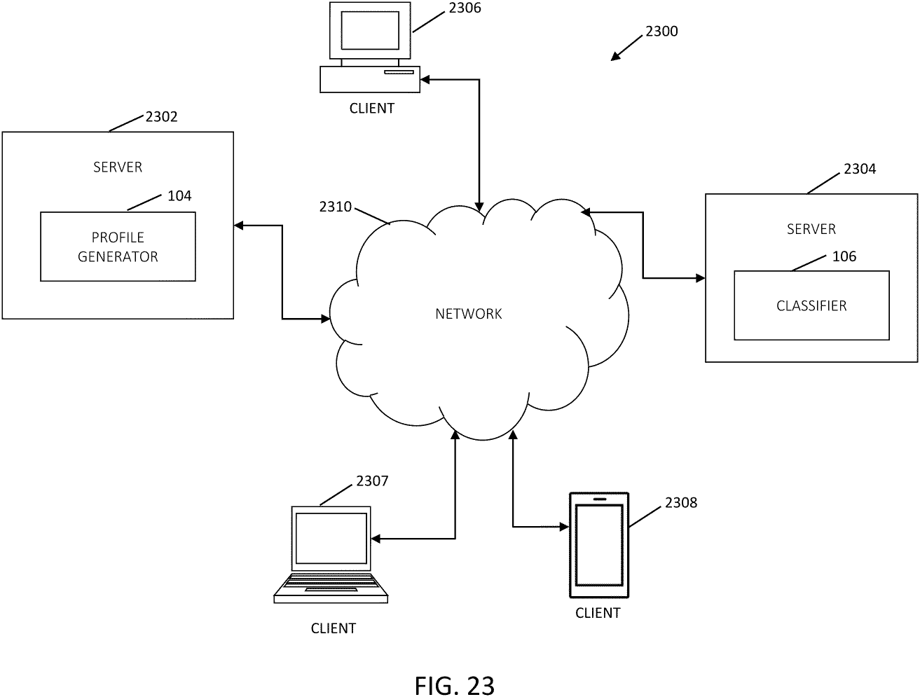

FIG. 23 is a schematic diagram of an example distributed computing environment 2300 in which systems and/or methods described herein may be implemented in accordance with one or more embodiments. The environment 2300 may include client and server devices, such as two servers 2302 and 2304, and three clients 2306-2308, interconnected by one or more networks, such as network 2310. The devices of the environment 2300 may interconnect via wired connections, wireless connections, or a combination of wired and wireless connections. The servers 2302 and 2304 may include one or more devices capable of receiving, generating, storing, processing, executing, and/or providing information. For example, the servers 2302 and 2304 may include a computing device, such as a server, a desktop computer, a laptop computer, a tablet computer, a handheld computer, or a similar device.

The clients 2306-2308 may be capable of receiving, generating, storing, processing, executing, and/or providing information. Information may include any type of machine-readable information having substantially any format that may be adapted for use, e.g., in one or more networks and/or with one or more devices. The information may include digital information and/or analog information. The information may further be packetized and/or non-packetized. In an embodiment, the clients 2306-2308 may download data and/or code from the servers 2302 and 2304 via the network 2310. In some implementations, the client 2306 may be a desktop computer, the client 2307 may be a laptop computer, and the client 2308 may be a mobile phone, e.g., a smart phone. Nonetheless, it should be understood that any of the clients 2306-2308 may be desktop computers, workstations, laptop computers, tablet computers, handheld computers, mobile phones (e.g., smart phones, radiotelephones, etc.), electronic readers, or similar devices. In some implementations, the clients 2306-2308 may receive information from and/or transmit information to the servers 2302 and 2304.

The network 2310 may include one or more wired and/or wireless networks. For example, the network 2310 may include a cellular network, a public land mobile network (PLMN), a local area network (LAN), a wide area network (WAN), a metropolitan area network (MAN), a telephone network (e.g., the Public Switched Telephone Network (PSTN)), an ad hoc network, an intranet, the Internet, a fiber optic-based network, and/or a combination of these or other types of networks. Information may be exchanged between network devices using any network protocol, such as, but not limited to, the Internet Protocol (IP), Asynchronous Transfer Mode (ATM), Synchronous Optical Network (SONET), the User Datagram Protocol (UDP), Institute of Electrical and Electronics Engineers (IEEE) 802.11, etc.

The servers 2302 and 2304 may host applications or processes accessible by the clients 2306-2308. For example, the server 2302 may host the profile generator 104. The server 2304 may host the classifier 106.

The number of devices and/or networks shown in FIG. 23 is provided as an example. In practice, there may be additional devices and/or networks, fewer devices and/or networks, different devices and/or networks, or differently arranged devices and/or networks than those shown in FIG. 23. Furthermore, two or more devices shown in FIG. 23 may be implemented within a single device, or a single device shown in FIG. 23 may be implemented as multiple, distributed devices. Additionally, one or more of the devices of the distributed computing environment 2300 may perform one or more functions described as being performed by another one or more devices of the environment 2300.

The foregoing description of embodiments is intended to provide illustration and description, but is not intended to be exhaustive or to limit the disclosure to the precise form disclosed. Modifications and variations are possible in light of the above teachings or may be acquired from a practice of the disclosure. For example, while a series of acts has been described above with respect to the flow diagrams, the order of the acts may be modified in other implementations. In addition, the acts, operations, and steps may be performed by additional or other modules or entities, which may be combined or separated to form other modules or entities. Further, non-dependent acts may be performed in parallel. Also, the term “user”, as used herein, is intended to be broadly interpreted to include, for example, a computer or data processing system or a human user of a computer or data processing system, unless otherwise stated.

In some embodiments, a user may interact with the rating task 102, the profile generator 104, and/or the classifier 106 using spoken commands that may be input to the data processing system 2200 through a microphone or by using eye, hand, facial, or other body gestures that may be input to the data processing system 2200 through a camera. In addition, auditory outputs may be generated by the rating task 102, the profile generator 104, and/or the classifier 106 additionally or alternatively to the graphically presented outputs, and the auditory outputs may be presented to the user through a speaker.

Further, certain embodiments of the disclosure may be implemented as logic that performs one or more functions. This logic may be hardware-based, software-based, or a combination of hardware-based and software-based. Some or all of the logic may be stored in one or more tangible non-transitory computer-readable storage media and may include computer-executable instructions that may be executed by a computer or data processing system, such as system 2200. The computer-executable instructions may include instructions that implement one or more embodiments of the disclosure. The tangible non-transitory computer-readable storage media may be volatile or non-volatile and may include, for example, flash memories, dynamic memories, removable disks, and non-removable disks.

The following examples implement one or more aspects of methods and/or systems of the present disclosure. These examples are non-limiting examples. Features of different examples may be combined in other implementations. Features of each example may be modified or removed in other implementations.

Aspect 1. A computer-implemented method for predicting a diagnosis of a human subject, the method comprising: accessing rating information created by the human subject for a plurality of pictures that are organized into picture categories, wherein the rating information includes positive ratings and negative ratings of the plurality of pictures; determining, by one or more processors, from the rating information, approach ratings (K+) and avoidance ratings (K₋) for the picture categories; computing, by the one or more processors, for the picture categories, approach entropy values (H+), avoidance entropy values (H_), approach standard deviation values (σ₊), and avoidance standard deviation values (σ₋), from the approach ratings (K+), the avoidance ratings (K₋), the positive ratings, and the negative ratings; generating, by the one or more processors, a value function of the approach entropy values (H+) and the avoidance entropy values (H₋) as a function of the approach ratings (K+) and the avoidance ratings (K₋); generating, by the one or more processors, a limit function of the approach standard deviation values (σ₊) and the avoidance standard deviation values (σ₋) as a function of the approach ratings (K+) and the avoidance ratings (K₋); deriving one or more first judgment variables from the value function; deriving one or more second judgment variables from the limit function; applying the one or more first judgment variables and the one or more second judgment variables to a trained machine learning (ML) model or classifier; and generating, by the trained ML model or classifier, a diagnostic prediction of a neurological condition for the human subject based on the one or more first judgment variables and the one or more second judgment variables.

Aspect 2. The computer-implemented method of aspect 1 wherein the neurological condition for which the diagnostic prediction is generated is cognitive decline or a history of depression.

Aspect 3. The computer-implemented method of aspect 1 or 2 wherein trained ML model or classifier is a random forest classifier or a gaussian mixture model.

Aspect 4. The computer-implemented method of any of the preceding aspects wherein the one or more first judgment variables include one or more of: a risk aversion value based on (i) a ratio of a second derivative of the value function to a first derivative of the value function and (ii) a predetermined quantity of the approach ratings; a loss resilience value based on (i) an absolute value of the ratio of the second derivative of the value function to the first derivative of the value function and (ii) a predetermined quantity of the avoidance ratings; a loss aversion value based on an absolute value of a ratio of a linear regression slope of a logarithm of the avoidance ratings versus a logarithm of the avoidance entropy values to a linear regression slope of a logarithm of the approach ratings versus a logarithm of the approach entropy values; an ante value based on a positive offset of the approach ratings when the approach entropy values is zero; and an insurance value based on a negative offset of the avoidance ratings when the avoidance entropy values is zero.

Aspect 5. The computer-implemented method of any of the preceding aspects wherein the one or more second judgment variables include one or more of: a peak positive risk value based on a given value of the approach standard deviation values when a derivative of the approach standard deviation values to a derivative of the approach ratings

$$\left(\frac{d {σ}_{+}}{d {K}_{+}}\right)$$

is zero; a peak negative risk value based on a given value of the avoidance standard deviation values when a derivative of the avoidance standard deviation values to a derivative of the avoidance ratings

$$\left(\frac{d {σ}_{-}}{d {K}_{-}}\right)$$

is zero; a reward tipping point being a given value of the approach ratings when the derivative of the approach standard deviation values to the derivative of the approach ratings

$$\left(\frac{d {σ}_{+}}{d {K}_{+}}\right)$$

is zero; an aversion tipping point being a given value of the avoidance ratings when the derivative of the avoidance standard deviation to the derivative of the avoidance ratings

$$\left(\frac{d {σ}_{-}}{d {K}_{-}}\right)$$

is zero; a total reward risk value based an area under the limit function for the approach ratings and theapproach standard deviation values; and a total aversion risk based on an area under the limit function for the avoidance ratings and the avoidance standard deviation values.

Aspect 6. The computer-implemented method of any of the preceding aspects wherein the generating the value function includes applying a curve fitting tool to a plot of the approach entropy values (H+) and the avoidance entropy values (H₋) versus the approach ratings (K+) and the avoidance ratings (K₋).

Aspect 7. The computer-implemented method of any of the preceding aspects wherein the generating the limit function includes applying a curve fitting tool to a plot of the approach standard deviation values (σ₊) and the avoidance standard deviation values (σ₋) versus the approach ratings (K+) and the avoidance ratings (K₋).

Aspect 8. The computer-implemented method of any of the preceding aspects further comprising: generating a tradeoff function between the approach entropy values and the avoidance entropy values; and deriving one or more third judgment variables from the tradeoff function, wherein the applying further includes applying the one or more third judgment variables from the tradeoff function to the trained ML model and the prediction is further based on the third judgment variables.

Aspect 9. The computer-implemented method of aspect 8 wherein the one or more third judgment variables include one or more of: a reward-aversion tradeoff value based on a mean of polar angles of points on a plot of the tradeoff function; a tradeoff range value based on a standard deviation of the polar angles of the points on the plot of the tradeoff function; a reward-aversion consistency based on an average Euclidian distance of the points on the plot of the tradeoff function to an origin of the plot; and a consistency range value based on a standard deviation of radial distances of the points on the plot of the tradeoff function to the origin of the plot.

Aspect 10. The computer-implemented method of any of the preceding aspects wherein the pictures are presented to the human subject through a rating task running on a device.

Aspect 11. The computer-implemented method of any of the preceding aspects wherein the picture categories include one or more of sports, disasters, cute animals, aggressive animals, nature, and food.

Aspect 12. The computer-implemented method of any of the preceding aspects wherein the approach ratings (K+) are based on the averages (means) of the positive ratings for the picture categories and the avoidance ratings (K₋) are based on the averages (means) of the negative ratings for the picture categories.

Aspect 13. A computer-implemented method for predicting a diagnosis of a human subject, the method comprising: accessing rating information associated with the human subject for a plurality of evaluation items organized into categories, wherein the rating information includes positive ratings and negative ratings of the plurality of evaluation items; determining, by one or more processors, from the rating information, approach ratings (K+) and avoidance ratings (K₋) for the categories; computing, by the one or more processors, for the categories, approach entropy values (H+), avoidance entropy values (H_), approach standard deviation values (σ₊), and avoidance standard deviation values (σ₋), from the approach ratings (K+), the avoidance ratings (K₋), the positive ratings, and the negative ratings; generating, by the one or more processors, a value function of the approach entropy values (H+) and the avoidance entropy values (H₋) as a function of the approach ratings (K+) and the avoidance ratings (K₋); generating, by the one or more processors, a limit function of the approach standard deviation values (σ₊) and the avoidance standard deviation values (σ₋) as a function of the approach ratings (K+) and the avoidance ratings (K₋); deriving at least one judgment variable from the value function or the limit function; accessing one or more demographic features associated with the subject; applying the at least one judgment variable and the one or more demographic features to a trained machine learning (ML) classifier; and generating, by the trained ML classifier, a diagnostic prediction for the human subject based on the at least one judgment variables and the one or more demographic features.

Aspect 14. The computer-implemented method of aspect 13 wherein the plurality of evaluation items include at least one picture, video, or sound.

Aspect 15. A computer-implemented method comprising: accessing rating information associated with a human subject for a plurality of evaluation items, wherein the evaluation items are selected to elicit an emotional response in the human subject and are organized into categories corresponding to emotion or approach-avoidance and the rating information includes positive ratings and negative ratings of the plurality of evaluation items; determining, by one or more processors, from the rating information, average approach ratings (K+) and average avoidance ratings (K₋) for the categories; computing, by the one or more processors, for the categories, approach entropy values (H+), avoidance entropy values (H₋), approach standard deviation values (σ₊), and avoidance standard deviation values (σ₋), from the approach ratings (K+), the avoidance ratings (K₋), the positive ratings, and the negative ratings; generating, by the one or more processors, at least one of (i) a value function of the approach entropy values (H+) and the avoidance entropy values (H₋) as a function of the approach ratings (K+) and the avoidance ratings (K₋) or (ii) a limit function of the approach standard deviation values (σ₊) and the avoidance standard deviation values (σ₋) as a function of the approach ratings (K+) and the avoidance ratings (K₋); deriving at least one judgment variable from the value function or the limit function; applying the at least one judgment variable to a trained machine learning (ML) classifier; and generating, by the trained ML classifier, a prediction for the human subject based on the at least one judgment variable, wherein the prediction is for a disease or condition, a purchase of a product or service, or a financial behavior.

Aspect 16. The computer-implemented method of aspect 15 further comprising: accessing one or more demographic features associated with the subject, wherein the applying further includes applying the one or more demographic features and the generating is further based on the one or more demographic features.

Aspect 17. One or more computer-readable media comprising program instructions for execution by one or more processors, the program instructions instructing the one or more processors to perform operations according to the method of any one of the preceding claims.

No element, act, or instruction used herein should be construed as critical or essential to the disclosure unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

The foregoing description has been directed to specific embodiments of the present disclosure. It will be apparent, however, that other variations and modifications may be made to the described embodiments, with the attainment of some or all of their advantages. Therefore, it is the object of the appended claims to cover all such variations and modifications as come within the true spirit and scope of the disclosure.

What is claimed is:

1. A computer-implemented method for predicting a diagnosis of a human subject, the method comprising:
accessing rating information created by the human subject for a plurality of pictures that are organized into picture categories, wherein the rating information includes positive ratings and negative ratings of the plurality of pictures;
determining, by one or more processors, from the rating information, approach ratings and avoidance ratings for the picture categories;
computing, by the one or more processors, for the picture categories, approach entropy values, avoidance entropy values, approach standard deviation values, and avoidance standard deviation values, from the approach ratings, the avoidance ratings, the positive ratings, and the negative ratings;
generating, by the one or more processors, a value function of the approach entropy values and the avoidance entropy values as a function of the approach ratings and the avoidance ratings;
generating, by the one or more processors, a limit function of the approach standard deviation values and the avoidance standard deviation values as a function of the approach ratings and the avoidance ratings;
deriving one or more first judgment variables from the value function;
deriving one or more second judgment variables from the limit function;
applying the one or more first judgment variables and the one or more second judgment variables to a trained machine learning (ML) classifier; and
generating, by the trained ML classifier, a diagnostic prediction of a neurological condition for the human subject based on the one or more first judgment variables and the one or more second judgment variables.

2. The computer-implemented method of claim 1 wherein the neurological condition for which the diagnostic prediction is generated is cognitive decline or a history of depression.

3. The computer-implemented method of claim 1 wherein trained ML classifier is a random forest classifier or a gaussian mixture model.

4. The computer-implemented method of claim 1 wherein the one or more first judgment variables include one or more of:
a risk aversion value based on (i) a ratio of a second derivative of the value function to a first derivative of the value function and (ii) a predetermined quantity of the approach ratings;
a loss resilience value based on (i) an absolute value of the ratio of the second derivative of the value function to the first derivative of the value function and (ii) a predetermined quantity of the avoidance ratings;
a loss aversion value based on an absolute value of a ratio of a linear regression slope of a logarithm of the avoidance ratings versus a logarithm of the avoidance entropy values to a linear regression slope of a logarithm of the approach ratings versus a logarithm of the approach entropy values;
an ante value based on a positive offset of the approach ratings when the approach entropy values is zero; and
an insurance value based on a negative offset of the avoidance ratings when the avoidance entropy values is zero.

5. The computer-implemented method of claim 1 wherein the one or more second judgment variables include one or more of:
a peak positive risk value based on a given value of the approach standard deviation values when a derivative of the approach standard deviation values to a derivative of the approach ratings is zero;
a peak negative risk value based on a given value of the avoidance standard deviation values when a derivative of the avoidance standard deviation values to a derivative of the avoidance ratings is zero;
a reward tipping point being a given value of the approach ratings when the derivative of the approach standard deviation values to the derivative of the approach ratings is zero;
an aversion tipping point being a given value of the avoidance ratings when the derivative of the avoidance standard deviation to the derivative of the avoidance ratings is zero;
a total reward risk value based an area under the limit function for the approach ratings and the approach standard deviation values; and
a total aversion risk based on an area under the limit function for the avoidance ratings and the avoidance standard deviation values.

6. The computer-implemented method of claim 1 wherein the generating the value function includes applying a curve fitting tool to a plot of the approach entropy values and the avoidance entropy values versus the approach ratings and the avoidance ratings.

7. The computer-implemented method of claim 1 wherein the generating the limit function includes applying a curve fitting tool to a plot of the approach standard deviation values and the avoidance standard deviation values versus the approach ratings and the avoidance ratings.

8. The computer-implemented method of claim 1 further comprising:
generating a tradeoff function between the approach entropy values and the avoidance entropy values; and
deriving one or more third judgment variables from the tradeoff function,
wherein the applying further includes applying the one or more third judgment variables from the tradeoff function to the trained ML model and the prediction is further based on the third judgment variables.

9. The computer-implemented method of claim 8 wherein the one or more third judgment variables include one or more of:
a reward-aversion tradeoff value based on a mean of polar angles of points on a plot of the tradeoff function;
a tradeoff range value based on a standard deviation of the polar angles of the points on the plot of the tradeoff function;
a reward-aversion consistency based on an average Euclidian distance of the points on the plot of the tradeoff function to an origin of the plot; and
a consistency range value based on a standard deviation of radial distances of the points on the plot of the tradeoff function to the origin of the plot.

10. The computer-implemented method of claim 1 wherein the pictures are presented to the human subject through a rating task running on a device.

11. The computer-implemented method of claim 1 wherein the picture categories include one or more of sports, disasters, cute animals, aggressive animals, nature, and food.

12. A computer-implemented method for predicting a diagnosis of a human subject, the method comprising:
accessing rating information associated with the human subject for a plurality of evaluation items organized into categories, wherein the rating information includes positive ratings and negative ratings of the plurality of evaluation items;
determining, by one or more processors, from the rating information, approach ratings and avoidance ratings for the categories;
computing, by the one or more processors, for the categories, approach entropy values, avoidance entropy values, approach standard deviation values, and avoidance standard deviation values, from the approach ratings, the avoidance ratings, the positive ratings, and the negative ratings;
generating, by the one or more processors, a value function of the approach entropy values and the avoidance entropy values as a function of the approach ratings and the avoidance ratings;
generating, by the one or more processors, a limit function of the approach standard deviation values and the avoidance standard deviation values as a function of the approach ratings and the avoidance ratings;
deriving at least one judgment variable from the value function or the limit function;
accessing one or more demographic features associated with the subject;
applying the at least one judgment variable and the one or more demographic features to a trained machine learning (ML) classifier; and
generating, by the trained ML classifier, a diagnostic prediction for the human subject based on the at least one judgment variables and the one or more demographic features.

13. The computer-implemented method of claim 12 wherein the plurality of evaluation items include at least one picture, video, or sound.

14. A apparatus comprising:
one or more memories storing rating information associated with a human subject for a plurality of evaluation items organized into categories, wherein the rating information includes positive ratings and negative ratings of the plurality of evaluation items; and
one or more processors coupled to the one or more memories, the one or more processors configured to:
determine from the rating information, approach ratings and avoidance ratings for the categories;
compute, for the categories, approach entropy values, avoidance entropy values, approach standard deviation values, and avoidance standard deviation values, from the approach ratings, the avoidance ratings, the positive ratings, and the negative ratings;
generate a value function of the approach entropy values and the avoidance entropy values as a function of the approach ratings and the avoidance ratings;
generate a limit function of the approach standard deviation values and the avoidance standard deviation values as a function of the approach ratings and the avoidance ratings;
derive at least one judgment variable from the value function or the limit function;
access one or more demographic features associated with the subject;
apply the at least one judgment variable and the one or more demographic features to a trained machine learning (ML) classifier; and
generate, by the trained ML classifier, a diagnostic prediction for the human subject based on the at least one judgment variables and the one or more demographic features.
